(19) United States
(12) Patent Application Publication
(10) Pub. No.: US 2023/0083749 A1
(43) Pub. Date: Mar. 16, 2023

(54) PROPOFOL COMPOSITIONS AND METHODS OF USE

(71) Applicant: Epalex Corporation, Mountain View, CA (US)

(72) Inventors: Steven L. Krill, San Clemente, CA (US); Feng-Jing Chen, Irvine, CA (US)

(21) Appl. No.: 17/821,943

(22) Filed: Aug. 24, 2022

Related U.S. Application Data
(60) Provisional application No. 63/236,388, filed on Aug. 24, 2021.

(51) Int. Cl. A61K 31/05; A61K 47/22; A61K 47/26; A61K 47/34; A61K 47/10

(52) U.S. Cl. CPC A61K 31/05; A61K 47/22; A61K 47/26; A61K 47/34; A61K 47/10

(57) ABSTRACT

The present disclosure provides pharmaceutical compositions for oral administration of propofol as well as methods of treatment using such pharmaceutical compositions.

### CROSS-REFERENCE TO RELATED APPLICATION

This application claims the benefit of U.S. Provisional Application No. 63/236,388, filed Aug. 24, 2021, the entirety of which is incorporated by reference herein.

### TECHNICAL FIELD

The present disclosure provides pharmaceutical compositions for oral administration of propofol as well as methods of treatment using such pharmaceutical compositions.

### BACKGROUND

DIPRIVAN® is an intravenously administered general anesthetic and sedation drug for use in the induction and maintenance of anesthesia or sedation. The active ingredient in DIPRIVAN® is Propofol, i.e., 2,6-diisopropylphenol, which has the chemical structure:

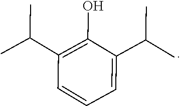

In addition to propofol, DIPRIVAN® also contains soybean oil (100 mg/mL), glycerol (22.5 mg/mL), egg lecithin (12 mg/mL); and disodium edetate (0.005%); with sodium hydroxide to adjust the pH to 7 to 8.5. See DIPRIVAN® prescribing information, 451094J/Revised: November 2017.

Expanded therapeutic use of propofol would benefit from the availability of an oral formulation of propofol. Oral administration of DIPRIVAN®, an oil-in-water emulsion, results in poor bioavailability of propofol, which has been attributed to limited aqueous solubility and extensive first pass metabolism by the liver. Thus, a need exists for an oral formulation of propofol that provides increased propofol bioavailabilty.

### SUMMARY

The present disclosure provides propofol microemulsion formulations that provide increased propofol oral bioavailability relative to that of DIPRIVAN®.

In some aspects, the disclosure provides pharmaceutical compositions comprising propofol and a surfactant, wherein the composition, when mixed for 30 minutes with water in a ratio of 0.1 grams composition to 10.0 mL water at 25° C., forms a mixture having a turbidity of less than 0.7 as measured by UV absorbance at 600 nm.

In other aspects, the disclosure provides pharmaceutical compositions comprising 10-30 wt % (w/w) of propofol; 40-80 wt % (w/w) of a surfactant; and 10-40 wt % (w/w) of a cosurfactant.

In other aspects, the disclosure provides pharmaceutical compositions comprising 10-25 wt % (w/w) of propofol; and 75-90 wt % (w/w) of a surfactant that is Vitamin E TPGS, polyoxyl 35 castor oil, or polysorbate 80, or a combination thereof.

In other aspects, the disclosure provides methods of treating diseases or disorders in a subject comprising orally administering to the subject a pharmaceutical composition of the disclosure.

### DETAILED DESCRIPTION OF ILLUSTRATIVE EMBODIMENTS

The present disclosure may be understood more readily by reference to the following detailed description of desired embodiments and the examples included therein. In the following specification and the claims that follow, reference will be made to a number of terms which have the following meanings.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one.

The term “subject,” is used herein to refer to an animal, for example a human, to whom treatment, including prophylactic treatment, with the pharmaceutical compositions or methods according to the present invention, is provided. The term “subject” as used herein refers to human and non-human animals.

As used herein, the term “treating” means reducing or eliminating the signs or symptoms of the condition for which propofol is being administered.

In some aspects, the disclosure is directed to pharmaceutical compositions comprising propofol; and a surfactant; wherein said composition, when mixed for 30 minutes with water in a ratio of 0.1 grams composition to 10.0 mL water at 25° C., forms a mixture having a turbidity of less than 0.7 as measured by UV absorbance at 600 nm.

In some aspects, the compositions of the disclosure comprise propofol, i.e., 2,6-diisopropylphenol, which has the chemical structure:

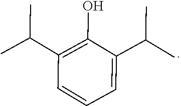

In some aspects, the compositions of the disclosure comprise up to 30% by weight (w/w) of propofol, such as for example, 30% by weight (w/w), 29% by weight (w/w), 28% by weight (w/w), 27% by weight (w/w), 26% by weight (w/w), 25% by weight (w/w), 24% by weight (w/w), 23% by weight (w/w), 22% by weight (w/w), 21% by weight (w/w), 20% by weight (w/w), 19% by weight (w/w), 18% by weight (w/w), 17% by weight (w/w), 16% by weight (w/w), 15% by weight (w/w), 14% by weight (w/w), 13% by weight (w/w), 12% by weight (w/w), 11% by weight (w/w), 10% by weight (w/w), 9% by weight (w/w), 8% by weight (w/w), 7% by weight (w/w), 6% by weight (w/w), 5% by weight (w/w), 4% by weight (w/w), 3% by weight (w/w), 2% by weight (w/w), 1% by weight (w/w), and the like. In some aspects, the compositions of the disclosure comprise more than 30% by weight (w/w) of propofol, such as for example, 31% by weight (w/w), 32% by weight (w/w), 33% by weight (w/w), 34% by weight (w/w), 35% by weight (w/w), 40% by weight (w/w), 45% by weight (w/w), 50% by weight (w/w), 55% by weight (w/w), 60% by weight (w/w), and the like.

In some embodiments, the compositions of the disclosure comprise at least 25% by weight (w/w) of propofol.

In some embodiments, the compositions of the disclosure comprise at least 20% by weight (w/w) of propofol.

In some embodiments, the compositions of the disclosure comprise at least 15% by weight (w/w) of propofol.

In some embodiments, the compositions of the disclosure comprise at least 14% by weight (w/w) of propofol.

In some embodiments, the compositions of the disclosure comprise at least 10% by weight (w/w) of propofol.

In some embodiments, the compositions of the disclosure comprise 25-30 wt. % (w/w) of propofol.

In some embodiments, the compositions of the disclosure comprise 20-30 wt. % (w/w) of propofol.

In some embodiments, the compositions of the disclosure comprise 15-30 wt. % (w/w) of propofol.

In some embodiments, the compositions of the disclosure comprise 14-30 wt. % (w/w) of propofol.

In some embodiments, the compositions of the disclosure comprise 13-30 wt. % (w/w) of propofol.

In some embodiments, the compositions of the disclosure comprise 12-30 wt. % (w/w) of propofol.

In some embodiments, the compositions of the disclosure comprise 11-30 wt. % (w/w) of propofol.

In some embodiments, the compositions of the disclosure comprise 10-30 wt. % (w/w) of propofol.

In some embodiments, the compositions of the disclosure comprise 25% by weight (w/w) of propofol.

In some embodiments, the compositions of the disclosure comprise 20% by weight (w/w) of propofol.

In some embodiments, the compositions of the disclosure comprise 15-20 wt. % (w/w) of propofol.

In some embodiments, the compositions of the disclosure comprise 14-20 wt. % (w/w) of propofol.

In some embodiments, the compositions of the disclosure comprise 13-20 wt. % (w/w) of propofol.

In some embodiments, the compositions of the disclosure comprise 12-20 wt. % (w/w) of propofol.

In some embodiments, the compositions of the disclosure comprise 11-20 wt. % (w/w) of propofol.

In some embodiments, the compositions of the disclosure comprise 10-20 wt. % (w/w) of propofol.

Compositions of the disclosure improve the dispersion of propofol in water such that an amount of a composition of the disclosure will have minimal turbidity in a volume of water, when tested according to the methods described herein. That is, compositions of the disclosure will not exhibit a turbid or milky appearance when tested according to the methods of the disclosure.

Several terms are used herein to qualitatively describe the visual appearance of mixtures. Generally speaking, these terms describe the relative amount of light that appears to transmit through the mixture. These terms, listed in order of decreasing light transmission, are transparent>hazy>turbid>milky. A “transparent” mixture is one that allows light to pass through such that objects placed behind the mixture can be distinctly seen. A “hazy” mixture is one that allows light to pass through only to the extent that objects placed behind the mixture are slightly obscured and made to appear vague or indistinct. A “turbid” mixture is one that is cloudy, opaque or thick with suspended matter. A “milky” mixture is one that appears like milk, and is not clear.

In some aspects, the compositions of the disclosure, when mixed for 30 minutes with water in a ratio of 0.1 grams composition to 10.0 mL water at 25° C., form a mixture having a turbidity of less than 0.7 as measured by UV absorbance at 600 nm, such as for example, a turbidity of less than 0.7, less than, 0.65, less than 0.6, less than 0.55, less than 0.5, less than 0.45, less than 0.4, less than 0.35, less than 0.3, less than 0.25, less than 0.2, less than 0.15, less than 0.1, and the like.

In some embodiments, the compositions of the disclosure, when mixed for 30 minutes with water in a ratio of 0.1 grams composition to 10.0 mL water at 25° C., form a mixture having a turbidity of less than 0.5 as measured by UV absorbance at 600 nm.

In other embodiments, the compositions of the disclosure, when mixed for 30 minutes with water in a ratio of 0.1 grams composition to 10.0 mL water at 25° C., form a mixture having a turbidity of less than 0.3 as measured by UV absorbance at 600 nm.

In some aspects, the compositions of the disclosure comprise a surfactant.

In some embodiments, the surfactant has a hydrophilic-lipophilic balance (HLB) of greater than or equal to 10, such as for example, 10, 11, 12, 13, 14, 15, 16, 17, 18, 19, or 20.

In some embodiments, the surfactant is Poloxamer 124; Polysorbate 80; Polyoxyl-8-glyceride; PEG-35 castor oil, or Vitamin E TPGS, or a combination thereof.

As used herein, Poloxamer 124 refers to polyoxyethylene-polyoxypropylene, such as, for example, that which is sold under the tradename Kollisolv® P124 Geismar.

As used herein Polysorbate 80 refers to polyoxyethylene-(20)sorbitanmonooleate, such as, for example, that which is sold under the tradename KOLLIPHOR PS80.

As used herein Polyoxyl-8-glyceride refers to caprylocaproyl polyoxyl-8 glycerides, such as, for example, that which is sold under the Labrasol ALF.

As used herein, PEG-35 castor oil refers to macrogolglycerol ricinoleate (or polyoxyl 35 castor oil), such as, for example, that sold under the tradename Kolliphor EL.

As used herein, Vitamin E TPGS refers to d-α-tocopheryl polyethylene glycol 1000 succinate.

In some embodiments, the surfactant is Vitamin E TPGS, PEG-35 castor oil, or polysorbate 80, or a combination thereof.

In some embodiments, the surfactant is Poloxamer 124.

In some embodiments, the surfactant is Polysorbate 80.

In some embodiments, the surfactant is Polyoxyl-8-glyceride.

In some embodiments, the surfactant is PEG-35 castor oil.

In some embodiments, the surfactant is Vitamin E TPGS.

The pharmaceutical compositions of the disclosure comprise up to 90% by weight (w/w) of the surfactant, such as, for example, 90% by weight (w/w), 85% by weight (w/w), 80% by weight (w/w), 75% by weight (w/w), 70% by weight (w/w), 65% by weight (w/w), 60% by weight (w/w), 55% by weight (w/w), 50% by weight (w/w), 45% by weight (w/w), 40% by weight (w/w), and the like.

In some embodiments, the compositions of the disclosure comprise at least 90% by weight (w/w) of the surfactant.

In some embodiments, the compositions of the disclosure comprise at least 85% by weight (w/w) of the surfactant.

In some embodiments, the compositions of the disclosure comprise at least 80% by weight (w/w) of the surfactant.

In some embodiments, the compositions of the disclosure comprise at least 75% by weight (w/w) of the surfactant.

In some embodiments, the compositions of the disclosure comprise at least 70% by weight (w/w) of the surfactant.

In some embodiments, the compositions of the disclosure comprise at least 65% by weight (w/w) of the surfactant.

In some embodiments, the compositions of the disclosure comprise at least 60% by weight (w/w) of the surfactant.

In some embodiments, the compositions of the disclosure comprise at least 55% by weight (w/w) of the surfactant.

In some embodiments, the compositions of the disclosure comprise at least 50% by weight (w/w) of the surfactant.

In some embodiments, the compositions of the disclosure comprise at least 45% by weight (w/w) of the surfactant.

In some embodiments, the compositions of the disclosure comprise at least 40% by weight (w/w) of the surfactant.

In some embodiments, the compositions of the disclosure comprise 80% by weight (w/w) of the surfactant.

In some embodiments, the compositions of the disclosure comprise 75-80 wt. % (w/w) of the surfactant.

In some embodiments, the compositions of the disclosure comprise 70-80 wt. % (w/w) of the surfactant.

In some embodiments, the compositions of the disclosure comprise 65-80 wt. % (w/w) of the surfactant.

In some embodiments, the compositions of the disclosure comprise 60-80 wt. % (w/w) of the surfactant.

In some embodiments, the compositions of the disclosure comprise 55-80 wt. % (w/w) of the surfactant.

In some embodiments, the compositions of the disclosure comprise 50-80 wt. % (w/w) of the surfactant.

In some embodiments, the compositions of the disclosure comprise 45-80 wt. % (w/w) of the surfactant.

In some embodiments, the compositions of the disclosure comprise up to 90% by weight (w/w) of the surfactant.

In some embodiments, the compositions of the disclosure comprise 85-90 wt. % (w/w) of the surfactant.

In some embodiments, the compositions of the disclosure comprise 80-90 wt. % (w/w) of the surfactant.

In some aspects, the pharmaceutical compositions of the disclosure further comprise a co-surfactant.

In some embodiments, the co-surfactant has a hydrophilic-lipophilic balance (HLB) of less than 10, such as, for example, 9, 8, 7, 6, 5, 4, 3, 2, and the like.

In some embodiments, the co-surfactant is oleoyl polyoxyl-6 glycerides, glyceryl monocaprylate, Glyceryl Caprylate/Caprate; Polyglyceryl-3 oleate; medium chain triglyceride (C8, C10); or a combination thereof.

As used herein, oleoyl polyoxyl-6 glycerides, includes, for example, the compound that is sold under the tradename LABRAFIL M1944CS.

As used herein, glyceryl monocaprylate includes, for example, the compound that is sold under the tradename CAPMUL 808G.

As used herein, glyceryl caprylate/caprate includes, for example, the compound that is sold under the tradename CAPMUL MCM.

As used herein, polyglyceryl-3 oleate includes, for example, the compound that is sold under the tradename PLUROL OLEIQUE CC497.

As used herein, medium chain triglyceride (C8, C10) includes, for example, the compound that is sold under the tradename CAPTEX 355.

In some embodiments, the co-surfactant is oleoyl polyoxyl-6 glycerides.

In some embodiments, the co-surfactant is glyceryl monocaprylate.

In some embodiments, the co-surfactant is glyceryl caprylate/caprate.

In some embodiments, the co-surfactant is polyglyceryl-3 oleate.

In some embodiments, the co-surfactant is medium chain triglyceride (C8, C10).

The pharmaceutical compositions of the disclosure comprise up to 40% by weight (w/w) of the co-surfactant, such as, for example, 40% by weight (w/w), 35% by weight (w/w), 30% by weight (w/w), 25% by weight (w/w), 20% by weight (w/w), 15% by weight (w/w), 10% by weight (w/w), and the like.

In some embodiments, the compositions of the disclosure comprise up to 35% by weight (w/w) of the co-surfactant.

In some embodiments, the compositions of the disclosure comprise at least 30% by weight (w/w) of the co-surfactant.

In some embodiments, the compositions of the disclosure comprise at least 25% by weight (w/w) of the co-surfactant.

In some embodiments, the compositions of the disclosure comprise at least 20% by weight (w/w) of the co-surfactant.

In some embodiments, the compositions of the disclosure comprise at least 15% by weight (w/w) of the co-surfactant.

In some embodiments, the compositions of the disclosure comprise at least 10% by weight (w/w) of the co-surfactant.

In some embodiments, the compositions of the disclosure comprise 40% by weight (w/w) of the co-surfactant.

In some embodiments, the compositions of the disclosure comprise 30-40 wt. % (w/w) of the co-surfactant.

In some embodiments, the compositions of the disclosure comprise 25-40 wt. % (w/w) of the co-surfactant.

In some embodiments, the compositions of the disclosure comprise 20-40 wt. % (w/w) of the co-surfactant.

In some embodiments, the compositions of the disclosure comprise 15-40 wt. % (w/w) of the co-surfactant.

In some embodiments, the compositions of the disclosure comprise 10-40 wt. % (w/w) of the co-surfactant.

In some aspects, the disclosure is directed to pharmaceutical compositions comprising 10-30 wt % (w/w) of propofol; 40-80 wt % (w/w) of a surfactant; and 10-40 wt % (w/w) of a cosurfactant.

In some embodiments, the compositions comprise 10-20 wt % (w/w) of propofol; 50-80 wt % (w/w) of PEG-35 castor oil; and 10-35 wt % (w/w) of glyceryl caprylate/caprate.

In some embodiments, the compositions comprise 20 wt % (w/w) of propofol; 60 wt % (w/w) of PEG-35 castor oil; and 20 wt % (w/w) of glyceryl caprylate/caprate.

In some embodiments, the compositions comprise 10-15 wt % (w/w) of propofol; 60-80 wt % (w/w) of PEG-35 castor oil; and 10-25 wt % (w/w) of oleoyl polyoxyl-6 glycerides.

In some embodiments, the compositions comprise 15 wt % (w/w) of propofol; 71 wt % (w/w) of PEG-35 castor oil; and 14 wt % (w/w) of oleoyl polyoxyl-6 glycerides.

In some embodiments, the compositions comprise 20-25 wt % (w/w) of propofol; 45-65 wt % (w/w) of PEG-35 castor oil; and 15-40 wt % (w/w) of glyceryl monocaprylate.

In some embodiments, the compositions comprise 25 wt % (w/w) of propofol; 50 wt % (w/w) of PEG-35 castor oil; and 25 wt % (w/w) of glyceryl monocaprylate.

In some embodiments, the compositions comprise 20 wt % (w/w) of propofol; 60 wt % (w/w) of PEG-35 castor oil; and 20 wt % (w/w) of glyceryl monocaprylate.

In some embodiments, the compositions comprise 20 wt % (w/w) of propofol; 55 wt % (w/w) of PEG-35 castor oil; and 25 wt % (w/w) of glyceryl monocaprylate.

In some embodiments, the compositions comprise 20 wt % (w/w) of propofol; 50 wt % (w/w) of PEG-35 castor oil; and 30 wt % (w/w) of glyceryl monocaprylate.

In some embodiments, the compositions comprise 20 wt % (w/w) of propofol; 45 wt % (w/w) of PEG-35 castor oil; and 35 wt % (w/w) of glyceryl monocaprylate.

In some aspects, the disclosure is directed to pharmaceutical compositions comprising 10-25 wt % (w/w) of propofol; and 75-90 wt % (w/w) of a surfactant that is Vitamin E TPGS, polyoxyl 35 castor oil, or polysorbate 80, or a combination thereof.

In some embodiments, these compositions comprise 20 wt % (w/w) propofol; and 80 wt % (w/w) of Vitamin E TPGS.

### Additional Excipients

In addition to the composition components described above, in some embodiments the compositions of the disclosure include additional components suitable for use in orally-administered drug products, such as, for example, water, glycols, oils, alcohols, flavoring agents, preservatives, coloring agents, or polymers.

The pharmaceutical compositions of the disclosure can be in any form suitable for oral (peroral) administration. Thus, the compositions of the disclosure can be in the form of a softgel capsule, a hard gelatin capsule, a HPMC capsule or any other non-gelatin-based capsule.

In some aspects, the compositions of the disclosure may be used in the treatment of diseases or disorders. In some aspects, the disclosure is directed to methods of treating a disease or disorder in subject in need thereof, the method comprising orally administering to said subject a pharmaceutical composition according to any one of the preceding claims that contains a therapeutically effective amount of propofol.

In other embodiments, a therapeutically effective amount of propofol is about 1 mg/kg to about 80 mg/kg, for example, an amount that is about (i.e., the specified number±10%) any one of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, or 80 mg/kg.

In some embodiments of the methods of the disclosure, the disease or disorder is migraine, acute repetitive seizures, seizure clusters, neuropathic pain, postherpetic neuralgia, traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, smoking cessation, tremor, essential tremor, Parkinsonian tremor (tremor in persons with Parkinson's disease), orthostatic tremor, primary writing tremor, cerebellar tremor, rubral tremor, neuropathic tremor or dystonic tremor.

In some embodiments, the disclosure is directed to the following aspects

Aspect 1. A pharmaceutical composition comprising
(a) propofol; and
(b) a surfactant;
   wherein said composition, when mixed for 30 minutes with water in a ratio of 0.1 grams composition to 10.0 mL water at 25° C., forms a mixture having a turbidity of less than 0.7 as measured by UV absorbance at 600 nm.
      Aspect 2. The pharmaceutical composition according to aspect 1, wherein said composition, when mixed for 30 minutes with water in a ratio of 0.1 grams composition to 10.0 mL water at 25° C., forms a mixture having a turbidity of less than 0.5 as measured by UV absorbance at 600 nm.
      Aspect 3. The pharmaceutical composition according to aspect 1, wherein said composition, when mixed for 30 minutes with water in a ratio of 0.1 grams composition to 10.0 mL water at 25° C., forms a mixture having a turbidity of less than 0.3 as measured by UV absorbance at 600 nm.
      Aspect 4. The pharmaceutical composition according to any one of the preceding aspects, wherein said composition further comprises a cosurfactant.
      Aspect 5. The pharmaceutical composition according to any one of the preceding aspects, wherein said surfactant has a hydrophilic-lipophilic balance (HLB) of greater than or equal to 10.
      Aspect 6. The pharmaceutical composition according to any one of the preceding aspects, wherein said surfactant is Poloxamer 124, Polysorbate 80, Polyoxyl-8-glyceride, PEG-35 castor oil, or Vitamin E TPGS, or a combination thereof.
      Aspect 7. The pharmaceutical composition according to aspect 6, wherein said surfactant is Vitamin E TPGS, PEG-35 castor oil, or polysorbate 80, or a combination thereof.
      Aspect 8. The pharmaceutical composition according to aspect 6, wherein said surfactant is Poloxamer 124.
      Aspect 9. The pharmaceutical composition according to aspect 6, wherein said surfactant is Polysorbate 80.
      Aspect 10. The pharmaceutical composition according to aspect 6, wherein said surfactant is Polyoxyl-8-glyceride.
      Aspect 11. The pharmaceutical composition according to aspect 6, wherein said surfactant is PEG-35 castor oil.
      Aspect 12. The pharmaceutical composition according to aspect 6, wherein said surfactant is Vitamin E TPGS.
      Aspect 13. The pharmaceutical composition according to any one of the preceding aspects, wherein the composition comprises up to 90% by weight (w/w) of the surfactant.
      Aspect 14. The pharmaceutical composition according to any one of the preceding aspects, wherein the composition comprises at least 85% by weight (w/w) of the surfactant.
      Aspect 15. The pharmaceutical composition according to any one of the preceding aspects, wherein the composition comprises at least 80% by weight (w/w) of the surfactant.
      Aspect 16. The pharmaceutical composition according to any one of the preceding aspects, wherein the composition comprises at least 75% by weight (w/w) of the surfactant.
      Aspect 17. The pharmaceutical composition according to any one of the preceding aspects, wherein the composition comprises at least 70% by weight (w/w) of the surfactant.
      Aspect 18. The pharmaceutical composition according to any one of the preceding aspects, wherein the composition comprises at least 65% by weight (w/w) of the surfactant.
      Aspect 19. The pharmaceutical composition according to any one of the preceding aspects, wherein the composition comprises at least 60% by weight (w/w) of the surfactant.
      Aspect 20. The pharmaceutical composition according to any one of the preceding aspects, wherein the composition comprises at least 55% by weight (w/w) of the surfactant.
      Aspect 21. The pharmaceutical composition according to any one of the preceding aspects, wherein the composition comprises at least 50% by weight (w/w) of the surfactant.
      Aspect 22. The pharmaceutical composition according to any one of the preceding aspects, wherein the composition comprises at least 45% by weight (w/w) of the surfactant.
      Aspect 23. The pharmaceutical composition according to any one of the preceding aspects, wherein the composition comprises at least 40% by weight (w/w) of the surfactant
      Aspect 24. The pharmaceutical composition according to any one of aspects 4-23, wherein said co-surfactant has a hydrophilic-lipophilic balance (HLB) of less than 10.
      Aspect 25. The pharmaceutical composition according to aspect 24, wherein said co-surfactant is oleoyl polyoxyl-6 glycerides, glyceryl monocaprylate, glyceryl caprylate/caprate, polyglyceryl-3 oleate, or caprylic/capric triglyceride or a combination thereof.
      Aspect 26. The pharmaceutical composition according to aspect 25 wherein said co-surfactant is oleoyl polyoxyl-6 glycerides.
      Aspect 27. The pharmaceutical composition according to aspect 25, wherein said co-surfactant is glyceryl monocaprylate.
      Aspect 28. The pharmaceutical composition according to aspect 25, wherein said co-surfactant is glyceryl caprylate/caprate.
      Aspect 29. The pharmaceutical composition according to aspect 25, wherein said co-surfactant is polyglyceryl-3 oleate.
      Aspect 30. The pharmaceutical composition according to aspect 25, wherein said co-surfactant is caprylic/capric triglyceride.
      Aspect 31. The pharmaceutical composition according to any one of aspects 4-30, wherein the composition comprises up to 40% by weight (w/w) of the co-surfactant
      Aspect 32. The pharmaceutical composition according to any one of aspects 4-30, wherein the composition comprises up to 35% by weight (w/w) of the co-surfactant.
      Aspect 33. The pharmaceutical composition according to any one of aspects 4-30, wherein the composition comprises at least 30% by weight (w/w) of the co-surfactant.
      Aspect 34. The pharmaceutical composition according to any one of aspects 4-30, wherein the composition comprises at least 25% by weight (w/w) of the co-surfactant.
      Aspect 35. The pharmaceutical composition according to any one of aspects 4-30, wherein the composition comprises at least 20% by weight (w/w) of the co-surfactant.
      Aspect 36. The pharmaceutical composition according to any one of aspects 4-30, wherein the composition comprises at least 15% by weight (w/w) of the co-surfactant.
      Aspect 37. The pharmaceutical composition according to any one of aspects 4-30, wherein the composition comprises at least 10% by weight (w/w) of the co-surfactant.
      Aspect 38. The pharmaceutical composition according to any one of the preceding aspects, wherein said composition comprises up to 30% by weight (w/w) of propofol.
      Aspect 39. The pharmaceutical composition according to any one of the preceding aspects, wherein said composition comprises at least 25% by weight (w/w) of propofol.
      Aspect 40. The pharmaceutical composition according to any one of the preceding aspects, wherein said composition comprises at least 20% by weight (w/w) of propofol.
      Aspect 41. The pharmaceutical composition according to any one of the preceding aspects, wherein said composition comprises at least 15% by weight (w/w) of propofol.
      Aspect 42. The pharmaceutical composition according to any one of the preceding aspects, wherein said composition comprises at least 14% by weight (w/w) of propofol.
      Aspect 43. The pharmaceutical composition according to any one of the preceding aspects, wherein said composition comprises at least 10% by weight (w/w) of propofol.
      Aspect 44. A pharmaceutical composition comprising
(a) 10-30 wt % (w/w) of propofol;
(b) 40-80 wt % (w/w) of a surfactant; and
(c) 10-40 wt % (w/w) of a cosurfactant.
   Aspect 45. The pharmaceutical composition according to aspect 44, wherein said composition comprises 25-30 wt. % (w/w) of propofol.
   Aspect 46. The pharmaceutical composition according to aspect 44, wherein said composition comprises 20-30 wt. % (w/w) of propofol.
   Aspect 47. The pharmaceutical composition according to aspect 44, wherein said composition comprises 15-30 wt. % (w/w) of propofol.
   Aspect 48. The pharmaceutical composition according to aspect 44, wherein said composition comprises 14-30 wt. % (w/w) of propofol.
   Aspect 49. The pharmaceutical composition according to aspect 44, wherein said composition comprises 13-30 wt. % (w/w) of propofol.
   Aspect 50. The pharmaceutical composition according to aspect 44, wherein said composition comprises 12-30 wt. % (w/w) of propofol.
   Aspect 51. The pharmaceutical composition according to aspect 44, wherein said composition comprises 11-30 wt. % (w/w) of propofol.
   Aspect 52. The pharmaceutical composition according to aspect 44, wherein said composition comprises 10-30 wt. % (w/w) of propofol.
   Aspect 53. The pharmaceutical composition according to any one of aspects 44-52, wherein said surfactant has a hydrophilic-lipophilic balance (HLB) of greater than or equal to 10.
   Aspect 54. The pharmaceutical composition according to any one of aspects 44-53, wherein said surfactant is Poloxamer 124, Polysorbate 80, Polyoxyl-8-glyceride, PEG-35 castor oil, or Vitamin E TPGS, or a combination thereof.
   Aspect 55. The pharmaceutical composition according to aspect 54, wherein said surfactant is Poloxamer 124.
   Aspect 56. The pharmaceutical composition according to aspect 54, wherein said surfactant is Polysorbate 80.
   Aspect 57. The pharmaceutical composition according to aspect 54, wherein said surfactant is Polyoxyl-8-glyceride.
   Aspect 58. The pharmaceutical composition according to aspect 54, wherein said surfactant is PEG-35 castor oil.
   Aspect 59. The pharmaceutical composition according to aspect 54, wherein said surfactant is Vitamin E TPGS.
   Aspect 60. The pharmaceutical composition according to any one of aspects 44-59, wherein the composition comprises 80% by weight (w/w) of the surfactant.
   Aspect 61. The pharmaceutical composition according to any one of aspects 44-59, wherein the composition comprises 75-80 wt. % (w/w) of the surfactant.
   Aspect 62. The pharmaceutical composition according to any one aspects 44-59, wherein the composition comprises 70-80 wt. % (w/w) of the surfactant.
   Aspect 63. The pharmaceutical composition according to any one of aspects 44-59, wherein the composition comprises 65-80 wt. % (w/w) of the surfactant.
   Aspect 64. The pharmaceutical composition according to any one of aspects 44-59, wherein the composition comprises 60-80 wt. % (w/w) of the surfactant.
   Aspect 65. The pharmaceutical composition according to any one of aspects 44-59, wherein the composition comprises 55-80 wt. % (w/w) of the surfactant.
   Aspect 66. The pharmaceutical composition according to any one of aspects 44-59, wherein the composition comprises 50-80 wt. % (w/w) of the surfactant.
   Aspect 67. The pharmaceutical composition according to any one of aspects 44-59, wherein the composition comprises 45-80 wt. % (w/w) of the surfactant.
   Aspect 68. The pharmaceutical composition according to any one of aspects 44-59, wherein said co-surfactant has a hydrophilic-lipophilic balance (HLB) of less than 10.
   Aspect 69. The pharmaceutical composition according to any one of aspects 44-68, wherein said co-surfactant is oleoyl polyoxyl-6 glycerides, glyceryl monocaprylate, glyceryl caprylate/caprate, polyglyceryl-3-oleate, or caprylic/capric triglyceride, or a combination thereof.
   Aspect 70. The pharmaceutical composition according to aspect 69, wherein said co-surfactant is oleoyl polyoxyl-6-glycerides.
   Aspect 71. The pharmaceutical composition according to aspect 69, wherein said co-surfactant is glyceryl monocaprylate.
   Aspect 72. The pharmaceutical composition according to aspect 69, wherein said co-surfactant is glyceryl caprylate/caprate.
   Aspect 73. The pharmaceutical composition according to aspect 69, wherein said co-surfactant is polyglyceryl-3-oleate.
   Aspect 74. The pharmaceutical composition according to aspect 69, wherein said co-surfactant is caprylic/capric triglyceride.
   Aspect 75. The pharmaceutical composition according to any one of aspects 44-74, wherein the composition comprises 40% by weight (w/w) of the co-surfactant.
   Aspect 76. The pharmaceutical composition according to any one of any one of aspects 44-74, wherein the composition comprises 30-40 wt. % (w/w) of the co-surfactant.
   Aspect 77. The pharmaceutical composition according to any one of any one of aspects 44-74, wherein the composition comprises 25-40 wt. % (w/w) of the co-surfactant.
   Aspect 78. The pharmaceutical composition according to any one of any one of aspects 44-74, wherein the composition comprises 20-40 wt. % (w/w) of the co-surfactant.
   Aspect 79. The pharmaceutical composition according to any one of any one of aspects 44-74, wherein the composition comprises 15-40 wt. % (w/w) of the co-surfactant.
   Aspect 80. The pharmaceutical composition according to any one of any one of aspects 44-74, wherein the composition comprises 10-40 wt. % (w/w) of the co-surfactant.
   Aspect 81. The pharmaceutical composition according to aspect 44, comprising
(a) 10-20 wt % (w/w) of propofol;
(b) 50-80 wt % (w/w) of PEG-35 castor oil; and
(c) 10-35 wt % (w/w) of glyceryl caprylate/caprate.
   Aspect 82. The pharmaceutical composition according to aspect 81, comprising
(a) 20 wt % (w/w) of propofol;
(b) 60 wt % (w/w) of PEG-35 castor oil; and
(c) 20 wt % (w/w) of glyceryl caprylate/caprate.
   Aspect 83. The pharmaceutical composition according to aspect 44, comprising
(a) 10-15 wt % (w/w) of propofol;
(b) 60-80 wt % (w/w) of PEG-35 castor oil; and
(c) 10-25 wt % (w/w) of oleoyl polyoxyl-6 glycerides.
   Aspect 84. The pharmaceutical composition according to aspect 83, comprising
(a) 15 wt % (w/w) of propofol;
(b) 71 wt % (w/w) of PEG-35 castor oil; and
(c) 14 wt % (w/w) of oleoyl polyoxyl-6 glycerides.
   Aspect 85. The pharmaceutical composition according to aspect 44, comprising
(a) 20-25 wt % (w/w) of propofol;
(b) 45-65 wt % (w/w) of PEG-35 castor oil; and
(c) 15-40 wt % (w/w) of glyceryl monocaprylate.
   Aspect 86. The pharmaceutical composition according to aspect 85, comprising
(a) 25 wt % (w/w) of propofol;
(b) 50 wt % (w/w) of PEG-35 castor oil; and
(c) 25 wt % (w/w) of glyceryl monocaprylate.
   Aspect 87. The pharmaceutical composition according to aspect 85, comprising
(a) 20 wt % (w/w) of propofol;
(b) 60 wt % (w/w) of PEG-35 castor oil; and
(c) 20 wt % (w/w) of glyceryl monocaprylate.
   Aspect 88. The pharmaceutical composition according to aspect 85, comprising
(a) 20 wt % (w/w) of propofol;
(b) 55 wt % (w/w) of PEG-35 castor oil; and
(c) 25 wt % (w/w) of glyceryl monocaprylate.
   Aspect 89. The pharmaceutical composition according to aspect 85, comprising
(a) 20 wt % (w/w) of propofol;
(b) 50 wt % (w/w) of PEG-35 castor oil; and
(c) 30 wt % (w/w) of glyceryl monocaprylate.
   Aspect 90. The pharmaceutical composition according to aspect 85, comprising
(a) 20 wt % (w/w) of propofol;
(b) 45 wt % (w/w) of PEG-35 castor oil; and
(c) 35 wt % (w/w) of glyceryl monocaprylate.
   Aspect 91. A pharmaceutical composition comprising
(a) 10-25 wt % (w/w) of propofol; and
(b) 75-90 wt % (w/w) of a surfactant that is Vitamin E TPGS, polyoxyl 35 castor oil, or polysorbate 80, or a combination thereof.
   Aspect 92. The pharmaceutical composition according to aspect 91, wherein said composition comprises 25% by weight (w/w) of propofol.
   Aspect 93. The pharmaceutical composition according to aspect 91, wherein said composition comprises 20% by weight (w/w) of propofol.
   Aspect 94. The pharmaceutical composition according to aspect 91, wherein said composition comprises 15-20 wt. % (w/w) of propofol.
   Aspect 95. The pharmaceutical composition according to aspect 91, wherein said composition comprises 14-20 wt. % (w/w) of propofol.
   Aspect 96. The pharmaceutical composition according to aspect 91, wherein said composition comprises 13-20 wt. % (w/w) of propofol.
   Aspect 97. The pharmaceutical composition according to aspect 91, wherein said composition comprises 12-20 wt. % (w/w) of propofol.
   Aspect 98. The pharmaceutical composition according to aspect 91, wherein said composition comprises 11-20 wt. % (w/w) of propofol.
   Aspect 99. The pharmaceutical composition according to aspect 91, wherein said composition comprises 10-20 wt. % (w/w) of propofol.
   Aspect 100. The pharmaceutical composition according to any one of aspects 91-99, wherein said surfactant is Polysorbate 80.
   Aspect 101. The pharmaceutical composition according to any one of aspects 91-99, wherein said surfactant is PEG-35 castor oil.
   Aspect 102. The pharmaceutical composition according to any one of aspects 91-99, wherein said surfactant is Vitamin E TPGS.
   Aspect 103. The pharmaceutical composition according to any one of any one of aspects 91-102, wherein the composition comprises up to 90% by weight (w/w) of the surfactant.
   Aspect 104. The pharmaceutical composition according to any one of aspects 91-102, wherein the composition comprises 85-90 wt. % (w/w) of the surfactant.
   Aspect 105. The pharmaceutical composition according to any one of aspects 91-102, wherein the composition comprises 80-90 wt. % (w/w) of the surfactant.
   Aspect 106. The pharmaceutical composition according to aspect 91, comprising
(a) 20 wt % (w/w) propofol;
(b) 80 wt % (w/w) of Vitamin E TPGS.
   Aspect 107. The pharmaceutical composition according to any one of aspects 44 to 106, wherein said composition, when mixed for 30 minutes with water in a ratio of 0.1 grams composition to 10.0 mL water at 25° C., forms a mixture having a turbidity of less than 0.7 as measured by UV absorbance at 600 nm.
   Aspect 108. The pharmaceutical composition according to aspect 107, wherein said composition, when mixed for 30 minutes with water in a ratio of 0.1 grams composition to 10.0 mL water at 25° C., forms a mixture having a turbidity of less than 0.5 as measured by UV absorbance at 600 nm.
   Aspect 109. The pharmaceutical composition according to aspect 107, wherein said composition, when mixed for 30 minutes with water in a ratio of 0.1 grams composition to 10.0 mL water at 25° C., forms a mixture having a turbidity of less than 0.3 as measured by UV absorbance at 600 nm.
   Aspect 110. The pharmaceutical composition according to any one of the preceding aspects in the form of a softgel capsule, a hard gelatin capsule, a HPMC capsule or any other non-gelatin-based capsule.
   Aspect 111. A method of treating a disease or disorder in subject in need thereof, said method comprising orally administering to said subject a pharmaceutical composition according to any one of the preceding aspects.
   Aspect 112. The method of aspect 111, wherein the disease or disorder is migraine, acute repetitive seizures, seizure clusters, neuropathic pain, postherpetic neuralgia, traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, smoking cessation, tremor, essential tremor, Parkinsonian tremor (tremor in persons with Parkinson's disease), orthostatic tremor, primary writing tremor, cerebellar tremor, rubral tremor, neuropathic tremor or dystonic tremor.

### EXAMPLES

### Preparation of Compositions

The excipients (surfactants and co-surfactants) shown in Table 1 are used in the experiments described below.

TABLE 1

| | | | | Physical State |
|---|---|---|---|---|
| | Excipient | Trade Name | Supplier | at RT (25° C.) |
| 1 | Poloxamer 124 | Kollisolv P124 | BASF | Liquid |
| | | Geismar | | |
| 2 | Polysorbate 80 | Kolliphor PS80 | BASF | Liquid |
| | (Polyoxyethylene(20)sorbitanmonooleate) | | | |
| 3 | Polyoxyl-8 glyceride (C8, C10) | Labrasol ALF | Gattefosse | Liquid |
| | (Caprylocaproyl Polyoxyl-8 glycerides) | | | |
| 4 | PEG-35 castor oil | Kolliphor EL | BASF | Liquid |
| 5 | Vitamin E TPGS | Tocophersolan | Antares | Solid |
| | (d-α-tocopheryl polyethylene glycol 1000 | | | |
| | succinate) | | | |
| 6 | Oleoyl polyoxyl-6 glycerides | Labrafil M1944CS | Gattefosse | Liquid |
| 7 | Polyglyceryl-3 oleate | Plural Oleique | Gattefosse | Liquid |
| | | CC497 | | |
| 8 | Glycerol fatty acid ester (mono, di- C8, | Capmul MCM | Abitec | Liquid/Solid |
| | C10) (Glyceryl Caprylate/Caprate) | | | |
| | (Medium Chain Mono- and Diglycerides) | | | |
| 9 | Propylene glycol fatty acid ester (mono-C8) | Capmul PG-8 | Abitec | Liquid |
| | (Propylene Glycol Monocaprylate) | | | |
| 10 | Medium chain triglyceride (C8, C10) | Captex 355 EP/NF | Abitec | Liquid |
| | (Caprylic/Capric Triglyceride) | | | |
| 11 | PEG-40 hydrogenated castor oil | Kolliphor RH40 | | Liquid/Solid |
| 12 | Glyceryl Monocaprylate | Capmul 808G | Abitec | |

Binary Mixture Experiments

Excipients that are liquid at room temperature are used directly for the miscibility study. Excipients that are not liquid at room temperature, Capmul MCM (not homogeneous at room temperature) and Vitamin E TPGS (Solid at Room temperature (20-25° C.), are kept in a water bath set at 50° C. to form a completely clear liquid, mixed to make it homogeneous, and dispensed for the experiment.

For each excipient, seven combinations of propofol and excipient are prepared as per Table 2 below.

TABLE 2

| Propofol/ | | | |
|---|---|---|---|
| Excipient | Propofol | Propofol | Excipient |
| Ratio | Concentration | Weight | Weight |
| (w/w) | (% w/w) | (g) | (g) |
| 2:1 | 67 | 1.0 | 0.5 |
| 1:1 | 50 | 1.0 | 1.0 |
| 1:2 | 33 | 1.0 | 2.0 |
| 1:3 | 25 | 1.0 | 3.0 |
| 1:4 | 20 | 0.5 | 2.0 |
| 1:6 | 14 | 0.5 | 3.0 |
| 1:9 | 10 | 0.5 | 4.5 |

To a clear centrifuge tube with Teflon-lining cap, the individual excipients are added first (amounts as per Table 2) then propofol is added. The weights of each component and the initial appearances of the mixtures are recorded. The tubes are placed on an end-over-end rotator for 24 hours at RT for mixing. The appearance of the mixture after mixing for 24 hours are recorded. No further mixing is employed. An additional physical appearance observation is made at a later time.

Vitamin E TPGS is not a liquid at room temperature. The excipient is gradually heated to˜50° C. to form a clear liquid, mixed, and the required amounts of the excipient is transferred to a clear centrifuge tube (per Table 2). It is observed that the excipient solidified as soon as it is transferred to the centrifuge tube. The required amount of Propofol is added to each tube (per Table 2). The weights of excipient and Propofol added to each tube is recorded. Tubes are placed on an end-over-end rotator for 24 hours at RT for mixing. Undissolved material is seen in each of the tubes. Mixing is continued for an additional 24 hours (total of 48 hours). A clear solution is formed in the tube with 2 parts of Propofol and 1 part of excipient. Undissolved material is seen in the rest. The tubes are mixed for an additional 24 hours at ˜50° C. Observations are recorded below in Table 16.

Capmul MCM is kept in the water bath set at 50° C. to form a clear liquid. Contents of the primary container are mixed to make it homogeneous. Prepared the sample as above (Section 3.3. to 3.4.)

Upon gentle mixing on an end-over-end rotator, the binary mixtures of propofol and Capmul PG-8, Labrafil M1944CS, Captex 355, Larasol ALF, Kollisolv P124, Kolliphor PS80, Capmul MCM or Kolliphor EL form clear, colorless, solutions at 2:1, 1:1, 1:2, 1:3, 1:4, 1:6 and 1:9 w/w ratios within 24 hours at room temperature.

Upon gentle mixing by an end-over-end rotator, the binary mixtures of Propofol and Plurol Oleique CC497 form clear, colorless, solutions at 2:1, 1:1 and 1:2 w/w ratios within 24 hours at room temperature. However, at 1:3, 1:4, 1:6 and 1:9 w/w ratios, the binary mixtures of Propofol and Plurol Oleique CC497 are hazy after gentle mixing on an end-over-end rotator for 48 hours at RT.

Upon gentle mixing by an end-over-end rotator, the binary mixtures of Propofol and vitamin E TPGS form a clear solution at 2.1 w/w ratios within 48 hours at room temperature. There are solids observed in the samples of the binary mixture sample of Propofol and vitamin E TPGS at 1:1, 1:2, 1:3, 1:4, 1:6 and 1:9 w/w ratio. After mixing at 50° C. for another 24 hours, these samples turn into clear solutions, but solids quickly reappear in some of the samples upon cooling to RT. At the 96 hour time point, i.e. 24 hours at RT following the end of the mixing at 50° C., only the binary mixture samples of Propofol and vitamin E TPGS at 2:1, 1:1 and 1:2 w/w ratio remain as clear solutions, free of solids.

The aqueous dispersibility test samples (n=1) are prepared by making a 100× w/v dilution of the following binary mixture sample of propofol and a hydrophilic surfactant excipient at 1:1, 1:2, 1:3, 1:4, 1:6 and 1:9 w/w ratios from the miscibility test with water as shown in Table 3 below:

TABLE 3

| | | | | 4 | |
|---|---|---|---|---|---|
| Propofol/ | 1 | 2 | 3 | PEG35 | 5 |
| Excipient | Labrasol | Poloxamer | Polysorbate | castor | Vitamin E |
| Ratio (wlw) | ALF | 124 | 80 | oil | TPGS |
| 2:1 | N/A | ✓ | N/A | N/A | N/A |
| 1:1 | ✓ | ✓ | ✓ | ✓ | ✓ |
| 1:2 | ✓ | ✓ | ✓ | ✓ | ✓ |
| 1:3 | ✓ | ✓ | ✓ | ✓ | ✓ |
| 1:4 | ✓ | ✓ | ✓ | ✓ | ✓ |
| 1:6 | N/A | N/A | ✓ | ✓ | ✓ |
| 1:9 | ✓ | N/A | ✓ | ✓ | ✓ |

N/A = no dilution is performed

Binary mixtures that are liquid at room temperature are directly used for the study. Binary mixture that are not liquid at room temperature (Propofol/Vitamin E TPGS at 1:3, 1:4, 1:6, and 1:9 (Solid at Room temperature (20-25° C.)) are kept in the water bath set at 50° C. to form a completely clear liquid, mixed to make it homogeneous, and dispensed the binary mixture for the experiment.

To a 20 mL scintillation vial, is added exactly 10.0 mL of water using an Eppendorf pipette.

The vial is placed on an analytical balance and the balance is tared.

100 mg of one binary mixture is dispensed directly into the tared vial.

Initial appearance of the mixture is recorded.

The scintillation vial gently swirled to mix the binary mixture with water for 3 minutes at RT. The appearance of the mixture is recorded.

The sample is placed on an end-over-end rotator and mixing is continued for 30 minutes at RT.

The appearance of the mixture is recorded.

Results are shown in Table 4 below:

TABLE 4

| Propofol | | | | | | |
|---|---|---|---|---|---|---|
| to | | | | | | |
| Excipient | | EXCIPIENT | | | | |
| Ratio | Time | | Kollisolv 124 (Poloxamer | | Kolliphor EL (polyoxyl | |
| (w/w) | point | Labrasol ALF | 124) | Polysorbate 80 | 35 castor oil) | Vitamin E TPGs |
| 2:1 | Initial | N/A | Oil droplets sink to the | N/A | N/A | N/A |
| | | | bottom | | | |
| | 3 min | N/A | Small oil droplets floating | N/A | N/A | N/A |
| | 30 mi | N/A | Hazy with small oil droplets | N/A | N/A | N/A |
| | | | floating | | | |
| 1:1 | Initial | White oil droplets | Oil droplets sink to the | White oil droplets | White oil droplets sink to | White oil droplets sink to |
| | | sink to the bottom | bottom | sink to the bottom | the bottom | the bottom |
| | 3 min | Hazy with small oil | Small oil droplets floating | Milky | Milky | Milky with white particles |
| | | droplets floating | | | | settled in the bottom |
| | 30 min | Hazy with small oil | Hazy with small oil droplets | Milky | Milky | Milky with white particles |
| | | droplets floating | floating | | | settled in the bottom |
| 1:2 | Initial | White oil droplets | White oil droplets sink to | White oil droplets | White mass stuck on the | White mass stuck at the |
| | | sink to the bottom | the bottom. | sink to the bottom | bottom | bottom |
| | 3 min | Milky with oil | Milky with white particles | Milky | Milky | Milky with white particles |
| | | droplets floating | settled in the bottom | | | settled in the bottom |
| | 30 min | Milky with oil | Milky with while particles | Milky | Milky | Milky with white particles |
| | | droplets floating | settled in the bottom | | | settled in the bottom |
| 1:3 | Initial | White oil droplets | White oil droplets sink to | White oil droplets | White mass stuck on the | White mass stuck on the |
| | | sink to the bottom | the bottom | sink to the bottom | bottom | bottom |
| | 3 min | Milky | Milky with white particles | Milky | Milky with small piece | Milky with white particles |
| | | | settled in the bottom | | of white mass stuck on | settled in the bottom |
| | | | | | the bottom | |
| | 30 mi | Milky | Milky with white particles | Milky | Milky | Hazy with white particles |
| | | | settled in the bottom | | | settled in the bottom |
| 1:4 | Initial | White oil droplets | While oil droplets sink to | White oil droplets | White mass stuck on the | White mass stuck on the |
| | | sink to the bottom | the bottom | sink to the bottom | bottom | bottom |
| | 3 min | Milky | Solution is milky with white | Milky | Milky with small piece | Milky with white particles |
| | | | particles settled in the | | of white mass stuck on | settled in tire bottom |
| | | | bottom | | the bottom | |
| | 30 min | Milky | Milky with white particles | Milky | Milky | Hazy with white particles |
| | | | settled in the bottom | | | settled in the bottom |
| 1:6 | Initial | N/A | N/A | Oily flat mass in tire | Oily flat mass in the | White gel like mass stuck |
| | | | | bottom | bottom | on the bottom |
| | 3 min | N/A | N/A | Hazy | Hazy with gel like mass | Hazy with gel like mass |
| | | | | | stuck on the bottom | stuck on the bottom |
| | 30 min | N/A | N/A | lazv | Hazy | Transparent w/bluish tint |
| 1:9 | Initial | White oil droplets in | N/A | Oily flat mass in the | Oily flat mass in the | Translucent, gel like mass |
| | | the bottom | | bottom | bottom | stuck on the bottom |
| | 3 min | Milky solution | N/A | Transparent with | Transparent with bluish | Translucent, wass stuck |
| | | | | bluish tint | tint | on the bottom |
| | 30 min | Milky solution | N/A | Transparent with | Transparent with bluish | Clear (but not as clear as |
| | | | | bluish tint | tint | water) |
| 1:9 repeat | Initial | N/A | N/A | Oily flat mass in the | Oily flat mass in the | Translucent, gel like mass |
| | | | | bottom | bottom | stuck on the bottom |
| | 3 min | N/A | N/A | Transparent with | Transparent with bluish | Transparent with bluish |
| | | | | bluish tint | tint, gel like mass stuck | tint, gel like wass stuck |
| | | | | | on the bottom | on the bottom |
| | 30 min | N/A | N/A | Transparent with | Transparent with bluish | Clear |
| | | | | bluish tint | tint | |

N/A: dilution was not performed

Ternary Mixture Experiments

Ternary mixture Nos. 1-45 are prepared using the binary mixtures from the miscibility test at the specified ratios with the co-surfactants Capmul MCM, Captex 355 and Labrafil M1944 CS as shown in Table 5 below.

Binary mixtures that are liquid at room temperature are directly used for the study.

Binary mixture that are not liquid at room temperature (Propofol/Vitamin E TPGS at 1:9, 1:6, 1:4 and 1:3 (Solid at Room temperature (20-25° C.)) are kept in a water bath set at 50° C. to form a completely clear liquid, mixed to make homogeneous, and dispensed for the experiment.

A 2 mL HPLC vial is placed on an analytical balance and the balance is tared.

To the vial is added an exactly weighed binary mixture using an Eppendorf pipette. The vial is capped with a PTFE-lined screw cap. The compositions of the ternary mixtures are shown in the Table 5 below.

The mixture is vortexed briefly and then continued mixing on an end-over-end rotator overnight until a homogeneous solution is formed at RT. The appearances of the resulting ternary mixtures is recorded and summarized in the Table 5 below.

Heated the mixture samples containing vitamin E TPGS at 50° C. briefly if necessary, to ensure the mixture samples is a single phase, clear liquid without any solids prior to dispensing and during mixing.

The aqueous dispersibility test samples are prepared by making a 100× w/v dilution of the ternary mixtures #1-45 in water. The appearance of each individual dispersion is recorded.

To each 20 mL scintillation vial, exactly 10.0 mL of water is added using an Eppendorf pipette.

The vial is placed on an analytical balance and the balance is tared.

100 mg of one ternary mixture is dispensed directly into the tared vial. The actual weight dispensed is recorded.

Initial appearances of the mixtures are recorded.

The scintillation vial is swirled gently to mix the ternary mixture with water for 3 minutes. The appearance of the mixture is recorded.

The sample is placed on an end-over-end rotator to continue the mixing for 30 minutes at RT. The appearance of the mixture is recorded.

The ternary mixture results are shown in Table 5 below.

TABLE 5

| | | Composition | | | | Description |
|---|---|---|---|---|---|---|
| | | of the | | | | of the |
| | | Ternary | | Description | Description | dispersion |
| | | Mixtures | | of the | of the | at the 30 |
| | | Based Upon | | ternary | dispersion | minutes |
| | | the Amount | Description | mixture | at the 3 | time point, |
| | | of the | of the | upon | minutes | end |
| Ternary | | Binary | ternary | dispensing | time point, | of end- |
| Mixture | Ternary Mixture | Mixtures | mixture | into the | end | over-end |
| No. | Composition (w/w) | (% w/w) | appearance | water | of swirling | mixing |
| | Propofol/Kolliphor | Propofol | | | | |
| | PS80/ | Kolliphor | | | | |
| | | PS80 | | | | |
| | Capmul MCM | Capmul | | | | |
| | (w/w) | MCM | | | | |
| 1 | 1:8:1 (10:80:10) | 10.1 | Solution is | Gel like | Hazy | Hazy |
| | | 80.1 | Clear and | mass sinks | | |
| | | 9.9 | homogenous | to the | | |
| | | | | bottom | | |
| 2 | 1:5:1 (14:72:14) | 14.4 | Solution is | Gel like | Milky | Milky |
| | | 71.3 | Clear and | mass sinks | | |
| | | 14.3 | homogenous | to the | | |
| | | | | bottom | | |
| 3 | 1:3:1 (20:60:20) | 20.2 | Solution is | Gel like | Milky | Milky |
| | | 60.0 | Clear and | mass sinks | | |
| | | 19.8 | homogenous | to the | | |
| | | | | bottom | | |
| 4 | 1:2:1 (25:50:25) | 25.1 | Solution is | Gel like | Milky | Milky |
| | | 49.9 | Clear and | mass sinks | | |
| | | 25.0 | homogenous | to the | | |
| | | | | bottom | | |
| 5 | 1:1:1 (33:34:33) | 33.5 | Solution is | White | Milky | Milky |
| | | 33.2 | Clear and | droplets | | |
| | | 33.3 | homogenous | sink to the | | |
| | | | | bottom | | |
| | Propofol/Kolliphor | Propofol | | | | |
| | EL/Capmul MCM | Kolliphor | | | | |
| | (w/w) | EL | | | | |
| | | Capmul | | | | |
| | | MCM | | | | |
| 6 | 1:8:1 (10:80:10) | 10.2 | Solution is | Gel like | Transparent | Transparent |
| | | 79.7 | Clear and | mass sink to | Bluish tint | Bluish tint |
| | | 10.1 | homogenous | the | | |
| | | | | bottom | | |
| 7 | 1:5:1 (14:72:14) | 14.4 | Solution is | Gel like | Hazy | Hazy |
| | | 71.2 | Clear and | mass sink to | | |
| | | 14.4 | homogenous | the | | |
| | | | | bottom | | |
| 8 | 1:3:1 (20:60:20) | 20.4 | Solution is | Gel like | Transparent | Transparent |
| | | 59.8 | Clear and | mass sink to | Bluish tint | Bluish tint |
| | | 19.8 | homogenous | the | | |
| | | | | bottom | | |
| 9 | 1:2:1 (25:50:25) | 25.1 | Solution is | White mass | Milky | Milky |
| | | 50.0 | Clear and | sink to the | | |
| | | 24.9 | homogenous | bottom | | |
| 10 | 1:1:1 (33:34:33) | 33.5 | Solution is | White mass | Milky | Milky |
| | | 33.2 | Clear and | sink to the | | |
| | | 33.3 | homogenous | bottom | | |
| | Propofol/Vitamin | Propofol | | | | |
| | E TPGS/ | Vitamin E | | | | |
| | Capmul MCM | TPGS | | | | |
| | (w/w) | Capmul | | | | |
| | | MCM | | | | |
| 11 | 1:8:1 (10:80:10) | 10.0 | Some TPGS | Gel like | Clear and | Transparent |
| | | 79.6 | is separated | mass sink to | Transparent | Bluish tint |
| | | 10.3 | as solid | the | with gel | |
| | | | | bottom | like mass | |
| | | | | | stuck to | |
| | | | | | the bottom | |
| 12 | 1:5:1 (14:72:14) | 14.4 | Some TPGS | Gel like | Hazy | Hazy |
| | | 71.5 | is separated | mass sink to | | |
| | | 14.2 | as solid | the | | |
| | | | | bottom | | |
| 13 | 1:3:1 (20:60:20) | 20.2 | Solution is | Gel like | Milky | Milky |
| | | 59.8 | Clear and | mass sink to | | |
| | | 20.0 | homogenous | the | | |
| | | | | bottom | | |
| 14 | 1:2:1 (25:50:25) | 25.1 | Solution is | Gel like | Milky | Milky |
| | | 50.1 | Clear and | droplets | | |
| | | 24.8 | homogenous | sink to the | | |
| | | | | bottom | | |
| 15 | 1:1:1 (33:34:33) | 33.3 | Solution is | Gel like | Milky | Milky |
| | | 33.4 | Clear and | droplets | | |
| | | 33.3 | homogenous | sink to the | | |
| | | | | bottom | | |
| | Propofol/Kolliphor | Propofol | | | | |
| | PS80/Captex 355 | Kolliphor | | | | |
| | (w/w) | PS80 | | | | |
| | | Captex 355 | | | | |
| 16 | 1:8:1 (10:80:10) | 10.0 | Solution is | Gel like | Hazy | Hazy |
| | | 79.9 | Clear and | mass sink to | | |
| | | 10.0 | homogenous | the | | |
| | | | | bottom | | |
| 17 | 1:5:1 (14:72:14) | 14.4 | Solution is | White mass | Turbid | Turbid |
| | | 71.3 | Clear and | settled in | | |
| | | 14.3 | homogenous | the | | |
| | | | | bottom | | |
| 18 | 1:3:1 (20:60:20) | 20.1 | Solution is | White mass | Milky | Milky |
| | | 59.9 | Clear and | settled in | | |
| | | 20.0 | homogenous | the | | |
| | | | | bottom | | |
| 19 | 1:2:1 (25:50:25) | 25.1 | Solution is | White mass | Milky | Milky |
| | | 49.8 | Clear and | settled in | | |
| | | 25.1 | homogenous | the | | |
| | | | | bottom | | |
| 20 | 1:1:1 (33:34:33) | 33.3 | Solution is | White oil | Turbid with | Turbid with |
| | | 33.3 | Clear and | droplets | white | white |
| | | 33.4 | homogenous | floating | particles | particles |
| | | | | on the | floating | floating |
| | | | | surface | | |
| | Propofol/Kolliphor | Propofol | | | | |
| | EL/Captex 355 | Kolliphor EL | | | | |
| | (w/w) | Captex 355 | | | | |
| 21 | 1:8:1 (10:80:10) | 10.2 | Solution is | Gel like | Transparent | Transparent |
| | | 79.4 | Clear and | mass | with Bluish | with Bluish |
| | | 10.4 | homogenous | floating on | tint | tint |
| | | | | the surface | | |
| 22 | 1:5:1 (14:72:14) | 14.4 | Solution is | Gel like | Hazy | Hazy |
| | | 70.4 | Clear and | mass | | |
| | | 15.2 | homogenous | floating on | | |
| | | | | the surface / | | |
| | | | | some stuck | | |
| | | | | on | | |
| | | | | the bottom | | |
| 23 | 1:3:1 (20:60:20) | 20.3 | Solution is | White mass | Milky | Milky |
| | | 59.7 | Clear and | settled in | | |
| | | 20.0 | homogenous | the | | |
| | | | | bottom | | |
| 24 | 1:2:1 (25:50:25) | 25.0 | Solution is | White mass | Milky | Milky |
| | | 49.9 | Clear and | settled in | | |
| | | 25.0 | homogenous | the | | |
| | | | | bottom | | |
| 25 | 1:1:1 (33:34:33) | 33.3 | Solution is | White mass | Milky with | Milky with |
| | | 33.2 | Clear and | settled in | white | white |
| | | 33.5 | homogenous | the | particles | particles |
| | | | | bottom | settled in | settled in |
| | | | | | the bottom | the bottom |
| | Propofol/Vitamin | Propofol | | | | |
| | E TPGS/Captex | Vitamin E | | | | |
| | 355 (w/w) | TPGS | | | | |
| | | Captex 355 | | | | |
| 26 | 1:8:1 (10:80:10) | 10.0 | Some TPGS | Oil droplets | Transparent | Transparent |
| | | 79.7 | is separated | floating on | with bluish | with bluish |
| | | 10.3 | as solid | the | tint | tint |
| | | | | surface | | |
| 27 | 1:5:1 (14:72:14) | 14.4 | Some TPGS | Gel like | Hazy | Hazy |
| | | 71.5 | is separated | mass stuck | | |
| | | 14.1 | as solid | at the | | |
| | | | | bottom | | |
| 28 | 1:3:1 (20:60:20) | 20.1 | Solution is | White gel | Milky | Milky |
| | | 59.9 | Clear and | like mass | | |
| | | 20.0 | homogenous | settled | | |
| | | | | in the | | |
| | | | | bottom | | |
| 29 | 1:2:1 (25:50:25) | 25.0 | Solution is | White gel | Milky | Milky |
| | | 50.1 | Clear and | like mass | | |
| | | 24.9 | homogenous | settled | | |
| | | | | in the | | |
| | | | | bottom | | |
| 30 | 1:1:1 (33:34:33) | 33.1 | Solution is | White oil | Milky with | Milky with |
| | | 33.3 | Clear and | droplets | white | white |
| | | 33.6 | homogenous | floating | particles | particles |
| | | | | on the | settled in | settled in |
| | | | | surface | the bottom | the bottom |
| | Propofol/Kolliphor | Propofol | | | | |
| | PS80/Labrafil | Kolliphor | | | | |
| | M1944CS (w/w) | PS80 | | | | |
| | | Labrafil | | | | |
| | | M1944CS | | | | |
| 31 | 1:8:1 (10:80:10) | 10.0 | Solution is | Gel like flat | Hazy | Hazy |
| | | 79.5 | Clear and | mass settled | | |
| | | 10.4 | homogenous | in | | |
| | | | | the bottom | | |
| 32 | 1:5:1 (14:72:14) | 14.4 | Solution is | Gel like | Milky | Milky |
| | | 71.5 | Clear and | mass in the | | |
| | | 14.2 | homogenous | bottom | | |
| 33 | 1:3:1 (20:60:20) | 20.1 | Solution is | White mass | Milky | Milky |
| | | 58.7 | Clear and | settled in | | |
| | | 21.3 | homogenous | the | | |
| | | | | bottom | | |
| 34 | 1:2:1 (25:50:25) | 25.0 | Solution is | White mass | Milky | Milky |
| | | 49.8 | Clear and | settled in | | |
| | | 25.2 | homogenous | the | | |
| | | | | bottom | | |
| 35 | 1:1:1 (33:34:33) | 33.4 | Solution is | White gel | Milky with | Milky with |
| | | 33.3 | Clear and | like | white | white |
| | | 33.3 | homogenous | droplets | particles | particles |
| | | | | floating on | floating | floating |
| | | | | the surface | | |
| | Propofol/Kolliphor | Propofol | | | | |
| | EL/ | Kolliphor | | | | |
| | Labrafil M1944CS | EL | | | | |
| | (w/w) | Labrafil | | | | |
| | | M1944CS | | | | |
| 36 | 1:8:1 (10:80:10) | 10.2 | Solution is | Gel like | Transparent | Transparent |
| | | 79.8 | Clear and | mass in the | with bluish | with bluish |
| | | 10.0 | homogenous | bottom | tint | tint |
| 37 | 1:5:1 (14:72:14) | 14.4 | Solution is | Gel like | Transparent | Transparent |
| | | 71.0 | Clear and | mass in the | with bluish | with bluish |
| | | 14.6 | homogenous | bottom | tint | tint |
| 38 | 1:3:1 (20:60:20) | 20.2 | Solution is | White mass | Turbid | Turbid |
| | | 58.7 | Clear and | settled in | | |
| | | 21.1 | homogenous | the | | |
| | | | | bottom | | |
| 39 | 1:2:1 (25:50:25) | 25.0 | Solution is | White mass | Milky | Milky |
| | | 49.8 | Clear and | settled in | | |
| | | 25.2 | homogenous | the | | |
| | | | | bottom | | |
| 40 | 1:1:1 (33:34:33) | 33.4 | Solution is | White mass | Milky with | Milky |
| | | 33.2 | Clear and | settled in | particles | |
| | | 33.4 | homogenous | the | floating | |
| | | | | bottom | | |
| | Propofol/Vitamin | Propofol | | | | |
| | E TPGS/ | Vitamin E | | | | |
| | Labrafil M1944CS | TPGS | | | | |
| | (w/w) | Labrafil | | | | |
| | | M1944CS | | | | |
| 41 | 1:8:1 (10:80:10) | 10.0 | Some TPGS | Oil droplets | Transparent | Transparent |
| | | 80.2 | is separated | floating on | Bluish tint | Bluish tint |
| | | 9.8 | as solid | the | | |
| | | | | surface | | |
| 42 | 1:5:1 (14:72:14) | 14.4 | Some TPGS | White gel | Hazy | Hazy |
| | | 71.3 | is separated | like mass | | |
| | | 14.4 | as solid | settled | | |
| | | | | in the | | |
| | | | | bottom | | |
| 43 | 1:3:1 (20:60:20) | 20.0 | Solution is | White gel | Milky | Milky |
| | | 59.6 | Clear and | like mass | | |
| | | 20.3 | homogenous | settled | | |
| | | | | in the | | |
| | | | | bottom | | |
| 44 | 1:2:1 (25:50:25) | 25.0 | Some TPGS | White gel | Milky | Milky |
| | | 49.6 | is separated | like mass | | |
| | | 25.4 | as solid | settled | | |
| | | | | in the | | |
| | | | | bottom | | |
| 45 | 1:1:1 (33:34:33) | 33.2 | Solution is | Yellow oil | Milky to | Milky to |
| | | 33.5 | Clear and | droplets | slightly | slightly |
| | | 33.3 | homogenous | floating | yellowish | yellowish |
| | | | | on the | | |
| | | | | surface | | |

Additional Ternary Mixture Experiments

The following ternary mixtures are prepared using the individual components at the specified ratios as shown in Table 6 below.

A 2 mL HPLC vial is placed on an analytical balance and the balance is tared.

An exactly weighed amount of the individual components is added to the vial using an Eppendorf pipette then the vial is closed with a PTFE-lined screw cap.

The mixture is vortexed briefly and then mixing on an end-over-end rotator is continued overnight until a homogeneous solution is formed at RT. The appearances of the resulting ternary mixture is recorded.

The mixtures containing Kolliphor RH40 are heated at 50° C., to ensure the mixture is a single phase, clear liquid without any solids prior to dispensing and during mixing.

The aqueous dispersibility test samples are prepared by making a 100× w/v dilution of the ternary mixtures (Group-1 to Group-4) in water. The appearance of the aqueous dispersion of the ternary mixtures is recorded.

To each 20 mL scintillation vial is added exactly 10.0 mL of water using an Eppendorf pipette.

The vial is placed on an analytical balance and the balance is tared.

100 mg of one ternary mixture is dispensed directly into the tared vial.

Initial appearances of the mixtures are recorded.

The scintillation vial is swirled gently to mix the ternary mixture with water for 3 minutes. The appearance of the mixture is recorded.

The sample is placed on an end-over-end rotator to the mixing is continued for 30 minutes at RT. The appearance of the mixture is recorded.

Samples 8-1 to 8-21 and 9-1 and 9-21

The ternary mixtures are prepared in 1 g quantity using individual components.

A 2 mL HPLC vial is placed on an analytical balance and the balance is tared.

To each vial is added an exactly weighed amount of the individual component using an Eppendorf pipette then the vial is closed with a PTFE-lined screw cap.

The mixture is vortexed briefly and then mixing is continued on an end-over-end rotator at 50° C. briefly until a homogeneous solution is formed (approximately 1 hour) and then cooled to RT for overnight mixing. The appearances of the resulting ternary mixtures are recorded.

The mixtures are heated at 50° C., to ensure the mixture samples is a single phase, clear liquid without any solid prior to dispensing and during mixing.

The aqueous dispersibility test samples are prepared by making a 100× w/v dilution of the ternary mixtures (Group-8 and Group-9) in water. The appearance of the aqueous dispersion of the ternary mixtures is recorded.

To each 20 mL scintillation vial is added exactly 10.0 mL of water using an Eppendorf pipette.

The vial is placed on an analytical balance and the balance is tared.

100 mg of one ternary mixture is dispensed directly into the tared vial.

Initial appearances of the mixtures are recorded.

The scintillation vial is swirled gently to mix the mixture with water for 3 minutes. The appearance is recorded.

The sample is placed on an end-over-end rotator and mixing is continued for 30 minutes at RT. The appearance is recorded.

TABLE 6

| | | | | | Description of |
|---|---|---|---|---|---|
| | | Appearance of | Description of | Description of | the dispersion |
| | | the ternary | the ternary | the dispersion | at the 30 minutes |
| | | mixture after | mixture upon | at the 3 minutes | time point, end |
| Ternary | Ternary Mixture | mixing at RT | dispensing | time point, end | of end-over-end |
| Mixture | Composition (w/w) | for 24 hr | into the water | of swirling | mixing |
| | Propofol/Kolliphor | | | | |
| | EL/Capmul MCM | | | | |
| 1-1 | 25:45:30 | A clear and | White mass settled in | Milky | Milky |
| | | homogeneous | the bottom | | |
| | | solution | | | |
| 1-2 | 25:50:25 | A clear and | White mass settled in | Milky | Milky |
| | | homogeneous | the bottom | | |
| | | solution | | | |
| 1-3 | 25:55:20 | A clear and | White mass settled in | Milky | Milky |
| | | homogeneous | the bottom | | |
| | | solution | | | |
| 1-4 | 25:60:15 | A clear and | White mass settled in | Milky | Milky |
| | | homogeneous | the bottom | | |
| | | solution | | | |
| 1-5 | 25:65:10 | A clear and | White mass settled in | Milky | Milky |
| | | homogeneous | the bottom | | |
| | | solution | | | |
| 1-6 | 30:40:30 | A clear and | White mass settled in | Milky | Milky |
| | | homogeneous | the bottom | | |
| | | solution | | | |
| 1-7 | 30:45:25 | A clear and | White mass settled in | Milky | Milky |
| | | homogeneous | the bottom | | |
| | | solution | | | |
| 1-8 | 30:50:20 | A clear and | White mass settled in | Milky | Milky |
| | | homogeneous | the bottom | | |
| | | solution | | | |
| 1-9 | 30:55:15 | A clear and | White mass settled in | Milky | Milky |
| | | homogeneous | the bottom | | |
| | | solution | | | |
| 1-10 | 30:60:10 | A clear and | White mass settled in | Milky | Milky |
| | | homogeneous | the bottom | | |
| | | solution | | | |
| 1-11 | 30:65:5 | A clear and | White mass settled in | Milky | Milky |
| | | homogeneous | the bottom | | |
| | | solution | | | |
| | Propofol/Kolliphor | | | | |
| | RH40/Capmul MCM | | | | |
| 2-1 | 25:45:30 | A clear and | White mass settled in | Milky | Milky |
| | | homogeneous | the bottom | | |
| | | solution | | | |
| 2-2 | 25:50:25 | A clear and | White mass settled in | Milky | Milky |
| | | homogeneous | the bottom | | |
| | | solution | | | |
| 2-3 | 25:55:20 | A clear and | White mass settled in | Milky | Milky |
| | | homogeneous | the bottom | | |
| | | solution | | | |
| 2-4 | 25:60:15 | A clear and | White mass settled in | Milky | Milky |
| | | homogeneous | the bottom | | |
| | | solution | | | |
| 2-5 | 25:65:10 | A clear and | White mass settled in | Milky | Milky |
| | | homogeneous | the bottom | | |
| | | solution | | | |
| 2-6 | 30:40:30 | A clear and | White mass settled in | Milky | Milky |
| | | homogeneous | the bottom | | |
| | | solution | | | |
| 2-7 | 30:45:25 | A clear and | White mass settled in | Milky | Milky |
| | | homogeneous | the bottom | | |
| | | solution | | | |
| 2-8 | 30:50:20 | A clear and | White mass settled in | Milky | Milky |
| | | homogeneous | the bottom | | |
| | | solution | | | |
| 2-9 | 30:55:15 | A clear and | White mass settled in | Milky | Milky |
| | | homogeneous | the bottom | | |
| | | solution | | | |
| 2-10 | 30:60:10 | A clear and | White mass settled in | Milky | Milky |
| | | homogeneous | the bottom | | |
| | | solution | | | |
| 2-11 | 20:60:20 | A clear and | White mass settled in | Milky | Milky |
| | | homogeneous | the bottom | | |
| | | solution | | | |
| | Propofol/Kolliphor | | | | |
| | EL/Labrafil M1944CS | | | | |
| 3-1 | 25:45:30 | A clear and | White mass settled in | Milky | Milky |
| | | homogeneous | the bottom | | |
| | | solution, | | | |
| | | yellow in color | | | |
| 3-2 | 25:50:25 | A clear and | White mass settled in | Milky | Milky |
| | | homogeneous | the bottom | | |
| | | solution, | | | |
| | | yellow in color | | | |
| 3-3 | 25:55:20 | A clear and | White mass settled in | Milky | Milky |
| | | homogeneous | the bottom | | |
| | | solution, | | | |
| | | yellow in color | | | |
| 3-4 | 25:60:15 | A clear and | White mass settled in | Milky | Milky |
| | | homogeneous | the bottom | | |
| | | solution | | | |
| 3-5 | 25:65:10 | A clear and | White mass settled in | Milky | Milky |
| | | homogeneous | the bottom | | |
| | | solution | | | |
| 3-6 | 30:40:30 | A clear and | White mass settled in | Milky | Milky |
| | | homogeneous | the bottom | | |
| | | solution | | | |
| 3-7 | 30:45:25 | A clear and | White mass settled in | Milky | Milky |
| | | homogeneous | the bottom | | |
| | | solution | | | |
| 3-8 | 30:50:20 | A clear and | White mass settled in | Milky | Milky |
| | | homogeneous | the bottom | | |
| | | solution | | | |
| 3-9 | 30:55:15 | A clear and | White mass settled in | Milky | Milky |
| | | homogeneous | the bottom | | |
| | | solution | | | |
| 3-10 | 30:60:10 | A clear and | White mass settled in | Milky | Milky |
| | | homogeneous | the bottom | | |
| | | solution | | | |
| 3-11 | 30:65: 5 | A clear and | White mass settled in | Milky | Milky |
| | | homogeneous | the bottom | | |
| | | solution | | | |
| | Propofol/Kolliphor | | | | |
| | RH40/Labrafil M1944CS | | | | |
| 4-1 | 25:45:30 | A clear and | White mass settled in | Milky | Milky |
| | | homogeneous | the bottom | | |
| | | solution | | | |
| 4-2 | 25:50:25 | A clear and | White mass settled in | Milky | Milky |
| | | homogeneous | the bottom | | |
| | | solution | | | |
| 4-3 | 25:55:20 | A clear and | White mass settled in | Milky | Milky |
| | | homogeneous | the bottom | | |
| | | solution | | | |
| 4-4 | 25:60:15 | A clear and | White mass settled in | Milky | Milky |
| | | homogeneous | the bottom | | |
| | | solution | | | |
| 4-5 | 25:65:10 | A clear and | White mass settled in | Milky | Milky |
| | | homogeneous | the bottom | | |
| | | solution | | | |
| 4-6 | 30:40:30 | A clear and | White mass settled in | Milky | Milky |
| | | homogeneous | the bottom | | |
| | | solution | | | |
| 4-7 | 30:45:25 | A clear and | White mass settled in | Milky | Milky |
| | | homogeneous | the bottom | | |
| | | solution | | | |
| 4-8 | 30:50:20 | A clear and | White mass settled in | Milky | Milky |
| | | homogeneous | the bottom | | |
| | | solution | | | |
| 4-9 | 30:55:15 | A clear and | White mass settled in | Milky | Milky |
| | | homogeneous | the bottom | | |
| | | solution | | | |
| 4-10 | 30:60:10 | A clear and | White mass settled in | Milky | Milky |
| | | homogeneous | the bottom | | |
| | | solution | | | |
| 4-11 | 20:60:20 | A clear and | White mass settled in | Milky | Milky |
| | | homogeneous | the bottom | | |
| | | solution | | | |
| | Propofol/Kolliphor | | | | |
| | EL/Capmul 808G (% w/w) | | | | |
| 8-1 | 20:65:15 | A clear and | White gel like mass | Hazy | Hazy |
| | | homogenous | settled in the bottom | | |
| | | solution | | | |
| 8-2 | 20:60:20 | A clear and | White gel like mass | Transparent | Transparent |
| | | homogenous | settled in the bottom | Bluish tint | Bluish tint |
| | | solution | | | |
| 8-3 | 20:55:25 | A clear and | White gel like mass | Transparent | Transparent |
| | | homogenous | settled in the bottom | Bluish tint | Bluish tint |
| | | solution | | | |
| 8-4 | 20:50:30 | A clear and | White gel like mass | Transparent | Transparent |
| | | homogenous | settled in the bottom | Bluish tint | Bluish tint |
| | | solution | | | |
| 8-5 | 20:45:35 | A clear and | White gel like mass | Transparent | Transparent |
| | | homogenous | settled in the bottom | Bluish tint | Bluish tint |
| | | solution | | | |
| 8-6 | 25:65:10 | A clear and | White gel like mass | Milky | Milky |
| | | homogenous | settled in the bottom | | |
| | | solution | | | |
| 8-7 | 25:60:15 | A clear and | White gel like mass | Milky | Milky |
| | | homogenous | settled in the bottom | | |
| | | solution | | | |
| 8-8 | 25:55:20 | A clear and | White gel like mass | Slightly | Slightly |
| | | homogenous | settled in the bottom | turbid with | turbid with |
| | | solution | | slight | slight |
| 8-9 | 25:50:25 | A clear and | White gel like mass | Transparent | Transparent |
| | | homogenous | settled in the bottom | Bluish tint | Bluish tint |
| | | solution | | | |
| 8-10 | 25:45:30 | A clear and | White gel like mass | Transparent | Less |
| | | homogenous | settled in the bottom | Bluish tint | transparent, |
| | | solution | | | bluish tint |
| 8-11 | 25:40:35 | A clear and | White gel like mass | Less | Milky |
| | | homogenous | settled in the bottom | transparent, | |
| | | solution | | bluish tint | |
| 8-12 | 25:35:40 | A clear and | White gel like mass | Milky | Milky |
| | | homogenous | settled in the bottom | | |
| | | solution | | | |
| 8-13 | 25:30:45 | A clear and | White gel like mass | Milky | Milky |
| | | homogenous | settled in the bottom | | |
| | | solution | | | |
| 8-14 | 30:65:5 | A clear and | White gel like mass | Milky | Milky |
| | | homogenous | settled in the bottom | | |
| | | solution | | | |
| 8-15 | 30:60:10 | A clear and | White gel like mass | Milky | Milky |
| | | homogenous | settled in the bottom | | |
| | | solution | | | |
| 8-16 | 30:55:15 | A clear and | White gel like mass | Milky | Milky |
| | | homogenous | settled in the bottom | | |
| | | solution | | | |
| 8-17 | 30:50:20 | A clear and | White gel like mass | Milky | Milky |
| | | homogenous | settled in the bottom | | |
| | | solution | | | |
| 8-18 | 30:45:25 | A clear and | White gel like mass | Milky | Milky |
| | | homogenous | settled in the bottom | | |
| | | solution | | | |
| 8-19 | 30:40:30 | A clear and | White gel like mass | Milky | Milky |
| | | homogenous | settled in the bottom | | |
| | | solution | | | |
| 8-20 | 30:35:35 | A clear and | White gel like mass | Milky | Milky |
| | | homogenous | settled in the bottom | | |
| | | solution | | | |
| 8-21 | 30:30:40 | A clear and | White gel like mass | Milky | Milky |
| | | homogenous | settled in the bottom | | |
| | | solution | | | |
| | propofol/Kolliphor | | | | |
| | EL/Plurol Oleique | | | | |
| | CC497 (% w/w) | | | | |
| 9-1 | 20:65:15 | A clear and | White gel like mass | Milky | Milky |
| | | homogenous | settled in the bottom | | |
| | | solution | | | |
| 9-2 | 20:60:20 | A clear and | White gel like mass | Turbid | Turbid |
| | | homogenous | settled in the bottom | | |
| | | solution | | | |
| 9-3 | 20:55:25 | A clear and | White gel like mass | Less turbid | Less turbid |
| | | homogenous | settled in the bottom | | with slight |
| | | solution | | | bluish tint |
| 9-4 | 20:50:30 | A clear and | White gel like mass | Turbid | Less turbid |
| | | homogenous | settled in the bottom | | |
| | | solution | | | |
| 9-5 | 20:45:35 | A clear and | White gel like mass | Milky | Milky |
| | | homogenous | settled in the bottom | | |
| | | solution | | | |
| 9-6 | 25:65:10 | A clear and | White gel like mass | Milky | Milky |
| | | homogenous | settled in the bottom | | |
| | | solution | | | |
| 9-7 | 25:60:15 | A clear and | White gel like mass | Milky | Milky |
| | | homogenous | settled in the bottom | | |
| | | solution | | | |
| 9-8 | 25:55:20 | A clear and | White gel like mass | Milky | Milky |
| | | homogenous | settled in the bottom | | |
| | | solution | | | |
| 9-9 | 25:50:25 | A clear and | White gel like mass | Milky | Milky |
| | | homogenous | settled in the bottom | | |
| | | solution | | | |
| 9-10 | 25:45:30 | A clear and | White gel like mass | Milky | Milky |
| | | homogenous | settled in the bottom | | |
| | | solution | | | |
| 9-11 | 25:40:35 | A clear and | White gel like mass | Milky | Milky |
| | | homogenous | settled in the bottom | | |
| | | solution | | | |
| 9-12 | 25:35:40 | A clear and | White gel like mass | Milky | Milky |
| | | homogenous | settled in the bottom | | |
| | | solution | | | |
| 9-13 | 25:30:45 | A clear and | White gel like mass | Milky | Milky |
| | | homogenous | settled in the bottom | | |
| | | solution | | | |
| 9-14 | 30:65:5 | A clear and | White gel like mass | Milky | Milky |
| | | homogenous | settled in the bottom | | |
| | | solution | | | |
| 9-15 | 30:60:10 | A clear and | White gel like mass | Milky | Milky |
| | | homogenous | settled in the bottom | | |
| | | solution | | | |
| 9-16 | 30:55:15 | A clear and | White gel like mass | Milky | Milky |
| | | homogenous | settled in the bottom | | |
| | | solution | | | |
| 9-17 | 30:50:20 | A clear and | White gel like mass | Milky | Milky |
| | | homogenous | settled in the bottom | | |
| | | solution | | | |
| 9-18 | 30:45:25 | A clear and | White gel like mass | Milky | Milky |
| | | homogenous | settled in the bottom | | |
| | | solution | | | |
| 9-19 | 30:40:30 | A clear and | White gel like mass | Milky | Milky |
| | | homogenous | settled in the bottom | | |
| | | solution | | | |
| 9-20 | 30:35:35 | A clear and | White gel like mass | Milky | Milky |
| | | homogenous | settled in the bottom | | |
| | | solution | | | |
| 9-21 | 30:30:40 | A clear and | White gel like mass | Milky | Milky |
| | | homogenous | settled in the bottom | | |
| | | solution | | | |

Quaternary Mixture Experiments

Binary mixtures of Kolliphor EL/TPGS at 2:1, 1:1 and 1:2 w/w, ratios are prepared as follows. Results are shown in Table 7.

A 20 mL scintillation vial is placed on an analytical balance and the balance is tared.

To each vial is added an exactly weighed amount of the individual component using an Eppendorf pipette and then the vial is closed with a cap.

The mixture is vortexed briefly and then mixing is continued on an end-over-end rotator at 50° C. until a homogeneous solution is formed (approximately 1 hour) and then the solution is cooled to RT and mixing is continued (approximately 1 hour).

The mixture is heated at 50° C., to ensure the mixture is a single phase, clear liquid without any solid prior to dispensing and during mixing.

The quaternary mixtures are prepared in 1 g quantity using the binary mixtures directly and ensured 5% of that quantity can be dispensed accurately at the specified ratios. The appearance of the quaternary mixtures are recorded.

A 2 mL HPLC vial is placed on an analytical balance and the balance is tared.

An exactly weighed amount of the individual component is added using an Eppendorf pipette and then the vial is closed with a PTFE-lined screw cap. The mixture is vortexed briefly then continued mixing on an end-over-end rotator at 50° C. until a homogeneous solution is formed (approximately 1 hour) and then the mixture is cooled to RT for overnight mixing. The appearances of the resulted quaternary mixtures are recorded

The mixture containing Vitamin E TPGS are heated at 50° C., to ensure the mixture samples is a single phase, clear liquid without any solids prior to dispensing and during mixing.

Aqueous Dispersion of Quaternary Mixtures

The aqueous dispersibility test samples are prepared by making a 100× w/v dilution of the quaternary mixtures in water. The appearance of the aqueous dispersion of the quaternary mixtures is recorded.

Exactly 10.0 mL of water is added to a 20 mL scintillation vial, using an Eppendorf pipette.

The vial is placed on an analytical balance and the balance is tared.

100 mg of one quaternary mixture is dispensed directly into the tared vial.

The initial appearance of the mixture is recorded.

The scintillation vial is swirled gently to mix the quaternary mixture with water for 3 minutes. The appearance of the mixture is recorded.

The sample is placed on an end-over-end rotator to continue the mixing for 30 minutes at RT. The appearance of the mixture is recorded.

TABLE 7

| | | | | | Description of |
|---|---|---|---|---|---|
| | | Appearance of | Description of | Description of | the dispersion |
| | | the Quaternary | the Quaternary | the dispersion | at the 30 minutes |
| | | mixture after | mixture upon | at the 3 minutes | time point, end |
| Quaternary | Quaternary Mixture | mixing at RT | dispensing | time point, end | of end-over-end |
| Mixture | Composition (w/w) | for 24 hr | into the water | of swirling | mixing |
| | Propofol/Kolliphor EL/ | | | | |
| | TPGS/Capmul MCM | | | | |
| 5-1 | 20:40:20:20 | A clear and | White mass settled in | Milky | Milky |
| | | homogeneous | the bottom | | |
| | | solution | | | |
| 5-2 | 20:53:27:0 | A clear and | White mass settled in | Turbid | Turbid |
| | | homogeneous | the bottom | | |
| | | solution | | | |
| 5-3 | 25:30:15:30 | A clear and | White mass settled in | Milky | Milky |
| | | homogeneous | the bottom | | |
| | | solution | | | |
| 5-4 | 25:33:17:25 | A clear and | White mass settled in | Milky | Milky |
| | | homogeneous | the bottom | | |
| | | solution | | | |
| 5-5 | 25:37:18:20 | A clear and | White mass settled in | Milky | Milky |
| | | homogeneous | the bottom | | |
| | | solution | | | |
| 5-6 | 25:40:20:15 | A clear and | White mass settled in | Milky | Milky |
| | | homogeneous | the bottom | | |
| | | solution | | | |
| 5-7 | 25:43:22:10 | A clear and | White mass settled in | Milky | Milky |
| | | homogeneous | the bottom | | |
| | | solution | | | |
| 5-8 | 25:47:23:5 | A clear and | White mass settled in | Milky | Milky |
| | | homogeneous | the bottom | | |
| | | solution | | | |
| 5-9 | 25:50:25:0 | A clear and | White mass settled in | Milky | Milky |
| | | homogeneous | the bottom | | |
| | | solution | | | |
| 5-10 | 30:27:13:30 | A clear and | White mass settled in | Milky | Milky |
| | | homogeneous | the bottom | | |
| | | solution | | | |
| 5-11 | 30:30:15:25 | A clear and | White mass settled in | Milky | Milky |
| | | homogeneous | the bottom | | |
| | | solution | | | |
| 5-12 | 30:33:17:20 | A clear and | White mass settled in | Milky | Milky |
| | | homogeneous | the bottom | | |
| | | solution | | | |
| 5-13 | 30:37:18:15 | A clear and | White mass settled in | Milky | Milky |
| | | homogeneous | the bottom | | |
| | | solution | | | |
| 5-14 | 30:40:20:10 | A clear and | White mass settled in | Milky | Milky |
| | | homogeneous | the bottom | | |
| | | solution | | | |
| 5-15 | 30:43:22:5 | A clear and | White mass settled in | Milky | Milky |
| | | homogeneous | the bottom | | |
| | | solution | | | |
| | Propofol/Kolliphor EL/ | | | | |
| | TPGS/Capmul MCM (% w/w) | | | | |
| 6-1 | 20:30:30:20 | A clear and | White mass settled in | Milky | Milky |
| | | homogeneous | the bottom | | |
| | | solution | | | |
| 6-2 | 20:40:40:0 | A mixture of | White mass settled in | Turbid | Turbid |
| | | solids and liquids | the bottom | | |
| 6-3 | 25:22.5:22.5:30 | A clear and | White mass settled in | Milky | Milky |
| | | homogeneous | the bottom | | |
| | | solution | | | |
| 6-4 | 25:25:25:25 | A clear and | White mass settled in | Milky | Milky |
| | | homogeneous | the bottom | | |
| | | solution | | | |
| 6-5 | 25:27.5:27.5:20 | A clear and | White mass settled in | Milky | Milky |
| | | homogeneous | the bottom | | |
| | | solution | | | |
| 6-6 | 25:30:30:15 | A clear and | White mass settled in | Milky | Milky |
| | | homogeneous | the bottom | | |
| | | solution | | | |
| 6-7 | 25:32.5:32.5:10 | A clear and | White mass settled in | Milky | Milky |
| | | homogeneous | the bottom | | |
| | | solution | | | |
| 6-8 | 25:35:35:5 | A clear and | White mass settled in | Milky | Milky |
| | | homogeneous | the bottom | | |
| | | solution | | | |
| 6-9 | 25:37.5:37.5:0 | A mixture of | White mass settled in | Turbid | Turbid |
| | | solids and liquids | the bottom | | |
| 6-10 | 30:20:20:30 | A clear and | White mass settled in | Milky | Milky |
| | | homogeneous | the bottom | | |
| | | solution | | | |
| 6-11 | 30:22.5:22.5:25 | A clear and | White mass settled in | Milky | Milky |
| | | homogeneous | the bottom | | |
| | | solution | | | |
| 6-12 | 30:25:25:20 | A clear and | White mass settled in | Milky | Milky |
| | | homogeneous | the bottom | | |
| | | solution | | | |
| 6-13 | 30:27.5:27.5:15 | A clear and | White mass settled in | Milky | Milky |
| | | homogeneous | the bottom | | |
| | | solution | | | |
| 6-14 | 30:30:30:10 | A clear and | White mass settled in | Milky | Milky |
| | | homogeneous | the bottom | | |
| | | solution | | | |
| 6-15 | 30:32.5:32.5:5 | A clear and | White mass settled in | Milky | Milky |
| | | homogeneous | the bottom | | |
| | | solution | | | |
| | Propofol/Kolliphor EL/ | | | | |
| | TPGS/Capmul MCM (% w/w) | | | | |
| 7-1 | 20:20:40:20 | A clear and | White mass settled in | Milky | Milky |
| | | homogeneous | the bottom | | |
| | | solution | | | |
| 7-2 | 20:27:53:0 | A mixture of | White mass settled in | Turbid | Turbid |
| | | solids and liquids | the bottom | | |
| 7-3 | 25:15:30:30 | A clear and | White mass settled in | Milky | Milky |
| | | homogeneous | the bottom | | |
| | | solution | | | |
| 7-4 | 25:17:33:25 | A clear and | White mass settled in | Milky | Milky |
| | | homogeneous | the bottom | | |
| | | solution | | | |
| 7-5 | 25:18:37:20 | A clear and | White mass settled in | Milky | Milky |
| | | homogeneous | the bottom | | |
| | | solution | | | |
| 7-6 | 25:20:40:15 | A clear and | White mass settled in | Milky | Milky |
| | | homogeneous | the bottom | | |
| | | solution | | | |
| 7-7 | 25:22:43:10 | A clear and | White mass settled in | Milky | Milky |
| | | homogeneous | the bottom | | |
| | | solution | | | |
| 7-8 | 25:23:47:5 | A clear and | White mass settled in | Milky | Milky |
| | | homogeneous | the bottom | | |
| | | solution | | | |
| 7-9 | 25:25:50:0 | A mixture of | White mass settled in | Turbid | Turbid |
| | | solids and liquids | the bottom | | |
| 7-10 | 30:13:17:30 | A clear and | White mass settled in | Milky | Milky |
| | | homogeneous | the bottom | | |
| | | solution | | | |
| 7-11 | 30:15:30:25 | A clear and | White mass settled in | Milky | Milky |
| | | homogeneous | the bottom | | |
| | | solution | | | |
| 7-12 | 30:17:33:20 | A clear and | White mass settled in | Milky | Milky |
| | | homogeneous | the bottom | | |
| | | solution | | | |
| 7-13 | 30:18:33:15 | A clear and | White mass settled in | Milky | Milky |
| | | homogeneous | the bottom | | |
| | | solution | | | |
| 7-14 | 30:20:40:10 | A clear and | White mass settled in | Milky | Milky |
| | | homogeneous | the bottom | | |
| | | solution | | | |
| 7-15 | 30:22:43:5 | A clear and | White mass settled in | Milky | Milky |
| | | homogeneous | the bottom | | |
| | | solution | | | |

Turbidity Measurements

The turbidity of compositions of the disclosure is measured using a UV spectrophotometer. The UV absorbance of the dispersion samples at a given wavelength are used to index the light transmission and provide an indication of improved aqueous dispersibility of propofol for potential oral delivery. UV absorbance of aqueous dispersions (100× dilution in water at room temperature after 30 minutes of mixing) are shown in Table 8.

TABLE 8

| Group #1 | | Kolliphor | Capmul | UV Abs @ | UV Abs @ |
|---|---|---|---|---|---|
| (% w/w) | Propofol | EL | MCM | 400 nm | 600 nm |
| 1-1 | 25 | 45 | 30 | 2.976 | 1.159 |
| 1-2 | 25 | 50 | 25 | 3.308 | 1.474 |
| 1-3 | 25 | 55 | 20 | 2.139 | 0.625 |
| 1-4 | 25 | 60 | 15 | 1.881 | 0.662 |
| 1-12 | 20 | 50 | 30 | 0.260 | 0.049 |
| 1-13 | 20 | 55 | 25 | 0.171 | 0.034 |
| 8 | 20 | 60 | 20 | 0.241 | 0.079 |
| 1-14 | 20 | 65 | 15 | 1.662 | 0.946 |
| 1-15 | 20 | 70 | 10 | 2.561 | 2.252 |
| 1-16 | 15 | 50 | 35 | 0.427 | 0.174 |
| 1-17 | 15 | 55 | 30 | 0.275 | 0.096 |
| 1-18 | 15 | 60 | 25 | 0.319 | 0.117 |
| 1-19 | 15 | 65 | 20 | 0.352 | 0.132 |
| 1-20 | 15 | 70 | 15 | 0.702 | 0.347 |
| 1-21 | 15 | 75 | 10 | 1.038 | 0.678 |
| EL-1 | 10 | 90 | 0 | 0.212 | 0.086 |
| EL-2 | 15 | 85 | 0 | 0.715 | 0.554 |
| EL-3 | 20 | 80 | 0 | 2.642 | 2.412 |
| EL-4 | 25 | 75 | 0 | 3.285 | 3.082 |
| EL-5 | 30 | 70 | 0 | 3.378 | 3.231 |
| Group #3 | | Kolliphor | Labrafil | UV Abs @ | UV Abs @ |
| (% w/w) | Propofol | EL | M1944CS | 400 nm | 600 nm |
| 3-12 | 20 | 60 | 20 | 1.313 | 0.404 |
| 3-13 | 20 | 65 | 15 | 1.204 | 0.389 |
| 3-14 | 20 | 70 | 10 | 2.886 | 1.964 |
| 3-15 | 20 | 75 | 5 | 2.874 | 2.599 |
| 3-16 | 15 | 60 | 25 | 0.340 | 0.100 |
| 3-17 | 15 | 65 | 20 | 0.155 | 0.037 |
| 3-18 | 15 | 70 | 15 | 0.122 | 0.030 |
| 3-19 | 15 | 75 | 10 | 1.553 | 0.969 |
| 3-20 | 15 | 80 | 5 | 1.091 | 0.802 |
| 37 | 14.3 | 71.4 | 14.3 | 0.091 | 0.029 |
| Group #8* | | Kolliphor | Capmul | Abs @ | Abs @ |
| (% w/w) | Propofol | EL | 808G | 400 nm | 600 nm |
| 8-1 | 20 | 65 | 15 | 0.295 | 0.153 |
| 8-2 | 20 | 60 | 20 | 0.192 | 0.067 |
| 8-3 | 20 | 55 | 25 | 0.159 | 0.033 |
| 8-4 | 20 | 50 | 30 | 0.144 | 0.029 |
| 8-5 | 20 | 45 | 35 | 0.185 | 0.036 |
| 8-6 | 25 | 65 | 10 | 2.988 | 2.521 |
| 8-7 | 25 | 60 | 15 | 1.415 | 0.637 |
| 8-8 | 25 | 55 | 20 | 0.873 | 0.266 |
| Group #1 | | Kolliphor | Capmul | UV Abs @ | UV Abs @ |
| (% w/w) | Propofol | EL | MCM | 400 nm | 600 nm |
| 8-9 | 25 | 50 | 25 | 0.661 | 0.126 |
| 8-10 | 25 | 45 | 30 | 2.124 | 0.420 |
| 8-11 | 25 | 40 | 35 | 3.549 | 1.552 |
| 8-16 | 30 | 55 | 15 | 3.424 | 2.364 |
| 8-17 | 30 | 50 | 20 | 3.482 | 3.006 |
| 8-18 | 30 | 45 | 25 | 3.494 | 3.204 |
| 8-19 | 30 | 40 | 30 | 3.458 | 1.468 |
| 8-22 | 20 | 70 | 10 | 1.994 | 1.531 |
| 8-23 | 20 | 40 | 40 | 0.410 | 0.107 |
| | | | Plurol | | |
| Group #9 | | Kolliphor | Oleique | Abs @ | Abs @ |
| (% w/w) | Propofol | EL | CC479 | 400 nm | 600 nm |
| 9-2 | 20 | 60 | 20 | 2.286 | 1.002 |
| 9-3 | 20 | 55 | 25 | 1.079 | 0.356 |
| 9-4 | 20 | 50 | 30 | 1.809 | 0.714 |
| 9-8 | 25 | 55 | 20 | 3.233 | 2.793 |
| 9-9 | 25 | 50 | 25 | 3.107 | 2.009 |
| 9-22 | 15 | 65 | 20 | 1.207 | 0.527 |
| 9-23 | 15 | 60 | 25 | 1.515 | 0.697 |
| 9-24 | 15 | 55 | 30 | 2.016 | 0.941 |
| 9-25 | 15 | 50 | 35 | 2.498 | 1.241 |

What is claimed:

1. A pharmaceutical composition comprising
(a) propofol; and
(b) a surfactant;
wherein said composition, when mixed for 30 minutes with water in a ratio of 0.1 grams composition to 10.0 mL water at 25° C., forms a mixture having a turbidity of less than 0.7 as measured by UV absorbance at 600 nm.

2. The pharmaceutical composition according to claim 1, wherein said composition further comprises a cosurfactant.

3. The pharmaceutical composition according to claim 1, wherein said surfactant has a hydrophilic-lipophilic balance (HLB) of greater than or equal to 10.

4. The pharmaceutical composition according to claim 1, wherein said surfactant is Poloxamer 124, Polysorbate 80, Polyoxyl-8-glyceride, PEG-35 castor oil, or Vitamin E TPGS, or a combination thereof.

5. The pharmaceutical composition according claim 1, wherein the composition comprises up to 90% by weight (w/w) of the surfactant.

6. The pharmaceutical composition according to claim 1, wherein the composition comprises at least 40% by weight (w/w) of the surfactant

7. The pharmaceutical composition according to claim 2, wherein said co-surfactant has a hydrophilic-lipophilic balance (HLB) of less than 10.

8. The pharmaceutical composition according to claim 2, wherein said co-surfactant is oleoyl polyoxyl-6 glycerides, glyceryl monocaprylate, glyceryl caprylate/caprate, polyglyceryl-3 oleate, or caprylic/capric triglyceride or a combination thereof.

9. The pharmaceutical composition according to claim 1, wherein the composition comprises up to 40% by weight (w/w) of the co-surfactant

10. The pharmaceutical composition according to claim 1, wherein the composition comprises at least 10% by weight (w/w) of the co-surfactant.

11. A pharmaceutical composition comprising
(a) 10-30 wt % (w/w) of propofol;
(b) 40-80 wt % (w/w) of a surfactant; and
(c) 10-40 wt % (w/w) of a cosurfactant.

12. The pharmaceutical composition according to claim 11, wherein said surfactant has a hydrophilic-lipophilic balance (HLB) of greater than or equal to 10.

13. The pharmaceutical composition according to claim 11, wherein said surfactant is Poloxamer 124, Polysorbate 80, Polyoxyl-8-glyceride, PEG-35 castor oil, or Vitamin E TPGS, or a combination thereof.

14. The pharmaceutical composition according to claim 11, wherein said co-surfactant has a hydrophilic-lipophilic balance (HLB) of less than 10.

15. The pharmaceutical composition according to claim 11, wherein said co-surfactant is oleoyl polyoxyl-6 glycerides, glyceryl monocaprylate, glyceryl caprylate/caprate, polyglyceryl-3-oleate, or caprylic/capric triglyceride, or a combination thereof.

16. A pharmaceutical composition comprising
(a) 10-25 wt % (w/w) of propofol; and
(b) 75-90 wt % (w/w) of a surfactant that is Vitamin E TPGS, polyoxyl 35 castor oil, or polysorbate 80, or a combination thereof.

17. The pharmaceutical composition according to claim 16, wherein said composition, when mixed for 30 minutes with water in a ratio of 0.1 grams composition to 10.0 mL water at 25° C., forms a mixture having a turbidity of less than 0.7 as measured by UV absorbance at 600 nm.

18. The pharmaceutical composition according to claim 1 in the form of a softgel capsule, a hard gelatin capsule, a HPMC capsule or any other non-gelatin-based capsule.

19. A method of treating a disease or disorder in subject in need thereof, said method comprising orally administering to said subject a pharmaceutical composition according to claim 1.

20. The method of claim 19, wherein the disease or disorder is migraine, acute repetitive seizures, seizure clusters, neuropathic pain, postherpetic neuralgia, traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, smoking cessation, tremor, essential tremor, Parkinsonian tremor (tremor in persons with Parkinson's disease), orthostatic tremor, primary writing tremor, cerebellar tremor, rubral tremor, neuropathic tremor or dystonic tremor.
